(19) United States
(12) Patent Application Publication
(10) Pub. No.: US 2026/0263773 A1
(43) Pub. Date: Sep. 10, 2026

(54) PRIMING CAP

(71) Applicant: ICU Medical, Inc., San Clemente, CA (US)

(72) Inventors: Thomas F. Fangrow, Mission Viejo, CA (US); Erik Scott Shauver, Tustin, CA (US)

(21) Appl. No.: 19/675,281

(22) Filed: May 12, 2026

Related U.S. Application Data
(63) Continuation of application No. 16/717,199, filed on Dec. 17, 2019, now abandoned.
Application No. 16/717,199 is a continuation of application No. PCT/US2018/038625, filed on Jun. 20, 2018.
(60) Provisional application No. 62/523,199, filed on Jun. 21, 2017.

(51) Int. Cl. A61M 39/16; A61M 39/20; A61M 39/22

(52) U.S. Cl. CPC A61M 39/162; A61M 39/20; A61M 39/22; A61M 2039/205; A61M 2205/0205; A61M 2205/583; A61M 2205/7536

(57) ABSTRACT

A priming cap includes a valve activation member and an engagement member. The valve activation member can be configured to open and close a medical connector. The engagement member can secure the priming cap to the medical connector in a first position in which the medical connector remains closed.

### INCORPORATION BY REFERENCE

This application is a continuation application of application Ser. No. 16/717,199, filed on Dec. 17, 2019, entitled “PRIMING CAP,” which claims the benefit under 35 U.S.C. § 120 and 35 U.S.C. § 365(c) as a continuation of International Application No. PCT/US2018/038625, designating the United States, with an international filing date of Jul. 25, 2017, entitled “PRIMING CAP,” which claims the benefit of U.S. Provisional Patent Application No. 62/523,199, filed on Jun. 21, 2017, the entire contents of each of which are hereby incorporated by reference herein in its entirety, forming part of the present disclosure. Any feature, structure, material, method, or step that is described and/or illustrated in any embodiment in any of the foregoing provisional patent application can be used with or instead of any feature, structure, material, method, or step that is described and/or illustrated in the following paragraphs of this specification or the accompanying drawings.

### BACKGROUND

### Field of the Invention

This disclosure relates generally to priming caps, and specifically to priming caps for medical connectors.

### Description of the Related Art

Catheters are widely used to treat patients requiring a variety of medical procedures. Catheters can either be acute, or temporary, for short-term use or chronic for long-term treatment. Catheters are commonly inserted into central veins (such as the vena cava) from peripheral vein sites to provide access to a patient's vascular system.

Catheter connections, such as, for example, connections of catheters to dialysis machine tubing, to IV line tubing, to infusion ports and to catheter caps, which are used to seal the end of a catheter to protect the sterility of the catheter and prevent fluid loss and/or particle contamination, are most often made utilizing the medical industry's standardized Luer taper fittings. These fittings, which may either be male couplings or female couplings, include a tapered end of standardized dimensions. Coupling is made by the press-fit of mating parts. A threaded lock-fit or other type of securing mechanism is commonly utilized to ensure the integrity of the pressure fit of the Luer fittings. There are also other non-standard fittings that can be used to selectively couple multiple components together.

In order to maintain a barrier to bacteria, debris, and fluid leakage, female connectors often have been provided with closures, such as septa, flexible seals, or other impediments, at their mating ends. Moreover, in the busy environment of hospitals and other medical settings, health care providers must often quickly manipulate multiple medical implements with one hand, making it difficult to retrieve certain caps and rapidly attach them upon disengagement of medical connectors. In addition, connectors are often employed at the end of gravity-fed fluid sources such as IV bags. When the connectors and tubing are initially connected to such sources, they are generally empty (i.e., filled with air) and must be primed with fluid before they can be connected to a patient.

Typically, certain caps can be implemented to help quickly prime a connector and/or established catheter line to or from a patient. Medical connectors may also require cleaning and/or sanitization, among other procedures, before a cap or other device can be connected to the medical connector for priming the system. This can elevate a patient's risk for bloodstream infection by creating an opening for bacterial entry and/or increase the amount of time required to prime the system. Thus, conventional systems may increase the risk that air can be trapped in the line without being properly vented. For example, conventional systems may require additional solutions, such as saline or a medication, to prevent air from entering an IV line. Thus, a system may not be effectively primed and/or efficiently primed in a timely manner.

### SUMMARY

Disclosed are various embodiments of priming caps. It is contemplated that the features of the various embodiments disclosed herein are combinable to form additional embodiments. Such combinations are within the scope of this disclosure.

According to some embodiments, a priming cap includes a conduit housing and a resilient member. The conduit housing can define a valve activation member. The conduit housing can include an upper housing, a lower housing coupled with the upper housing, a fluid passageway extending through the upper housing and the lower housing, and a plug positioned within the upper housing. The plug can vent trapped air and inhibit fluid from passing between the lower housing and an exterior of the priming cap. The resilient member can be coupled to the upper housing and shaped to at least partially surround an interior volume. The interior volume can receive a portion of a medical connector. The resilient member can include a retaining feature extending laterally across a portion of the resilient member. The retaining feature can secure the priming cap to a portion of the medical connector.

In some embodiments, the upper housing has an outer diameter and the lower housing has an outer diameter. The outer diameter of the upper housing can be larger than the outer diameter of the lower housing. In some embodiments, the resilient member forms a partial-cylindrical shape that can surround at least a portion of the medical connector. In some embodiments, the retaining feature includes a bumper that extends outwardly from an interior surface of the resilient member. The bumper can secure the priming cap to a securement feature of the medical connector.

In some embodiments, the retaining feature secures the priming cap to the medical connector in a first position. In the first position, the valve activation member may not open a seal of the medical connector to establish a fluid flow path between the medical connector and the fluid passageway of the conduit housing. In some embodiments, the upper housing includes a venting notch formed within a side wall of the upper housing, and the venting notch can allow trapped air to be vented out of the priming cap. In some embodiments, the venting notch includes a venting hole passing between an interior of the upper housing and an exterior of the priming cap.

In some embodiments, the plug further comprises a hydrophobic filter. In some embodiments, the conduit housing further comprises a transition region between the upper housing and the lower housing. The transition region can define a platform configured to support the plug within the upper housing. In some embodiments, the resilient arm includes at least two resilient arms. In some embodiments, the resilient arm includes a base portion and a head portion. The base portion and the head portion can be integrally formed and shaped in an arrow configuration. The arrow configuration can indicate to a user a proper orientation of the priming cap relative to the connector. In some embodiments, the head portion includes an upper region and a lower region, the upper region has an upper width and the lower region has a lower width, and the lower width is less than the upper width. In some embodiments, the head portion includes a maximum head width and the base portion includes a maximum base width, and the maximum base width is approximately one-half the maximum head width. In some embodiments, the head portion includes a maximum head width and the base portion includes a maximum base width, and the maximum base width is approximately ⅛, ¼, ⅓, ⅔, and/or ¾ or greater than the maximum head width. In some embodiments, the head portion has a head height and the base portion has a base height, and the head height is approximately one-half the base height. In some embodiments, the head portion has a head height and the base portion has a base height, and the head height is approximately ⅛, ¼, ⅓, ⅔, and/or ¾ or greater than the base height.

In some embodiments, an outer wall of the lower housing is tapered inwardly away from the upper housing. In some embodiments, the priming cap includes a connecting member configured to couple the resilient member with the conduit housing. The connecting member can include a lateral portion and a vertical portion. The vertical portion can extend vertically along at least a portion of an interior surface of the resilient member. The connecting member can support the resilient member at a position spaced away from the conduit housing.

According to some embodiments, an automatic closed priming system includes the priming cap and the medical connector. In some embodiments, the medical connector includes a female needleless connector. In some embodiments, an automatic closed priming system includes the priming cap and an extension device that can be coupled with a catheter.

According to some embodiments, a method of priming a medical connector includes providing a pre-assembled priming cap and connector assembly including the priming cap and the medical connector comprising a seal and activating the priming cap including transitioning the priming cap from a first position to a second position, the transitioning comprising pressing the priming cap into the medical connector. In the first positon, the valve activation member of the priming cap may not open the seal of the medical connector. In the second position the valve activation member of the priming cap may open the seal of the medical connector to establish fluid communication between the medical connector and the priming cap.

According to some embodiments, a priming system includes a vent cap and a female medical connector, wherein the vent cap is coupled with the female medical connector such that a portion of the vent cap is secured within the female medical connector.

According to some embodiments, a priming cap includes a conduit housing defining a valve activation member including: a fluid passageway extending through the conduit housing, a filter membrane configured to vent trapped air, and a member coupled to the conduit housing and shaped to at least partially surround a portion of a medical connector.

According to some embodiments, a priming system includes a vent cap comprising: a priming activation portion, an external member, and a female medical connector, wherein the priming activation portion and the external member are configured to slidably engage the female medical connector such that the priming activation portion is configured to slide within at least a portion of the female medical connector, and the external member is configured to slide along at least a portion of an exterior wall of the female medical connector.

### BRIEF DESCRIPTION OF THE DRAWINGS

Embodiments will now be described with reference to the following drawings, which are provided by way of example, and not limitation. Like reference numerals indicate identical or functionally similar elements. The sizes and relative proportions of all components and features shown in the drawings form part of this disclosure but should not be interpreted to be part of a claim unless specifically included in such claim.

FIG. 1A illustrates a side view of an embodiment of a connector attached to tubing configured to be in fluid communication with a patient's vasculature. The connector is in a closed position.

FIG. 1B illustrates a side view of the connector of FIG. 1A in an opened configuration.

FIG. 1C illustrates a side cross-sectional view of the connector of FIG. 1A taken along line 1C-1C.

FIG. 1D illustrates a side cross-sectional view of the connector of FIG. 1B taken along line 1D-1D.

FIG. 2 illustrates a side cross-sectional view of an embodiment of a connector.

FIG. 3 illustrates a side cross-sectional view of an embodiment of a connector.

FIG. 4 illustrates a side cross-sectional view of the connector of FIG. 3 rotated 90 degrees along a longitudinal axis of the connector.

FIG. 5A is a schematic of an embodiment of a priming cap and a connector in a first position.

FIG. 5B is a schematic of the priming cap and the connector of FIG. 5A in a second position.

FIG. 6A illustrates a front perspective view of an embodiment of a priming cap.

FIG. 6B illustrates a front view of the priming cap of FIG. 6A.

FIG. 6C illustrates a side view of the priming cap of FIG. 6A.

FIG. 6D shows a front perspective cross-sectional view of the priming cap of FIG. 6C taken along line 6D-6D.

FIG. 6E shows a side perspective cross-sectional view of the priming cap of FIG. 6B taken along line 6E-6E.

FIG. 6F shows a front cross-sectional view of an embodiment of the priming cap of FIG. 6C taken along line 6D-6D including a plug.

FIG. 6G shows a side cross-sectional view of an embodiment of the priming cap of FIG. 6B taken along line 6E-6E including a plug.

FIG. 7A illustrates a front view of an embodiment of a priming cap.

FIG. 7B illustrates a top view of the priming cap of FIG. 7A.

FIG. 7C shows a front cross-sectional view of an embodiment of the priming cap of FIG. 7B taken along line 7C-7C.

FIG. 8A illustrates a front view of an embodiment of a priming cap and a connector in a first position.

FIG. 8B illustrates a front cross-sectional view of the priming cap and the connector of FIG. 8A taken along line 8B-8B.

FIG. 9A illustrates a front view of an embodiment of a priming cap and a connector in a second position.

FIG. 9B illustrates a front cross-sectional view of the priming cap and the connector of FIG. 9A taken along line 9B-9B.

FIG. 10 illustrates a front perspective view of an embodiment of a priming cap and a connector in a first position.

FIG. 11 illustrates a top view of an embodiment of a priming cap and a catheter line extension set.

FIG. 12A illustrates a front perspective view of an embodiment of a priming cap and a connector in a first position.

FIG. 12B illustrates a top view of the priming cap of FIG. 12A.

FIG. 12C illustrates a front view of the priming cap of FIG. 12A.

FIG. 13A illustrates a front perspective view of an embodiment of a priming cap and a connector in a first position.

FIG. 13B illustrates a side view of the priming cap of FIG. 13A.

FIG. 13C illustrates a top view of the priming cap of FIG. 13A.

FIG. 13D illustrates a front view of the priming cap of FIG. 13A.

FIG. 14A illustrates a front perspective view of an embodiment of a priming cap and a connector in a first position.

FIG. 14B illustrates a side view of the priming cap of FIG. 14A.

FIG. 14C illustrates a top view of the priming cap of FIG. 14A.

FIG. 14D illustrates a front view of the priming cap of FIG. 14A.

FIG. 15A illustrates a front perspective view of an embodiment of a priming cap.

FIG. 15B illustrates a side perspective view of the priming cap of FIG. 15A.

FIG. 15C illustrates a side view of the priming cap of FIG. 15A and a connector.

FIG. 16A illustrates a front perspective view of an embodiment of a priming cap.

FIG. 16B illustrates a partial side cross-sectional view of the priming cap of FIG. 10A and a connector.

FIG. 17 illustrates a front perspective view of an embodiment of a priming cap and a connector.

FIG. 18 illustrates a front cross-sectional view of an embodiment of a priming cap and a connector.

FIG. 19 illustrates a front cross-sectional view of an embodiment of a priming cap.

FIG. 20 illustrates a front cross-sectional view of an embodiment of a priming cap engaged with a front view of a connector.

FIG. 21A illustrates a front view of an embodiment of a priming cap and a connector in a first position.

FIG. 21B illustrates a front view of the priming cap and the connector of FIG. 21A in a second position.

FIG. 22 illustrates a front view of an embodiment of a priming cap and a connector a first position.

FIG. 23A illustrates a front view of an embodiment of a priming cap.

FIG. 23B illustrates a front view of the priming cap of FIG. 23A and a connector in a first position.

FIG. 23C illustrates a front view of the priming cap of FIG. 23A and a connector in a second position.

### DETAILED DESCRIPTION

Various systems, methods, and components can be used in different embodiments of the inventions. Some embodiments are illustrated in the accompanying figures; however, the figures are provided for convenience of illustration only, and should not be interpreted to limit the inventions to the particular combinations of features shown. Rather, any feature, structure, material, step, or component of any embodiment described and/or illustrated in this specification can be used by itself, or with or instead of any other feature, structure, material, step, or component of any other embodiment described and/or illustrated in this specification. Nothing in this specification is essential or indispensable. Any of the devices or connections or features that are described and/or illustrated anywhere in this specification can be configured to attach to, prime, protect, and/or sanitize luer connectors, which are in compliance with ISO standard 594 or ISO 80369, or can comply with any other industry standard that is applicable to medical fluid connectors.

This disclosure relates to embodiments of a priming cap that can be used to prime and/or protect medical connectors. A cap may be used with intravascular connectors associated with a fluid pathway, such as an IV line. All references to any type of connector (e.g., a female luer connector) in this application should be understood to include and disclose any type of medical implement that accomplishes or facilitates storage or transfer of medical fluid or connection of medical fluid lines (e.g., any open or resealable fluid line connector, syringe, catheter connector, vial, vial adapter, pump cartridge or disposable, pharmaceutical compounding component, female connector, blood-line connector, IV bag, catheter inserter, disinfectant cap, etc.).

Fluid pathways, once established, may provide direct access to a patient's blood stream and can be used intermittently to administer medications to a patient. These fluid pathways can have one or more associated medical connectors that can be connected to other medical connectors. In some embodiments, a plurality of corresponding connectors can have male and/or female connection regions, such as male and/or female luer connection regions or locks. The connection regions can provide a convenient way to connect and disconnect the fluid pathway at various times.

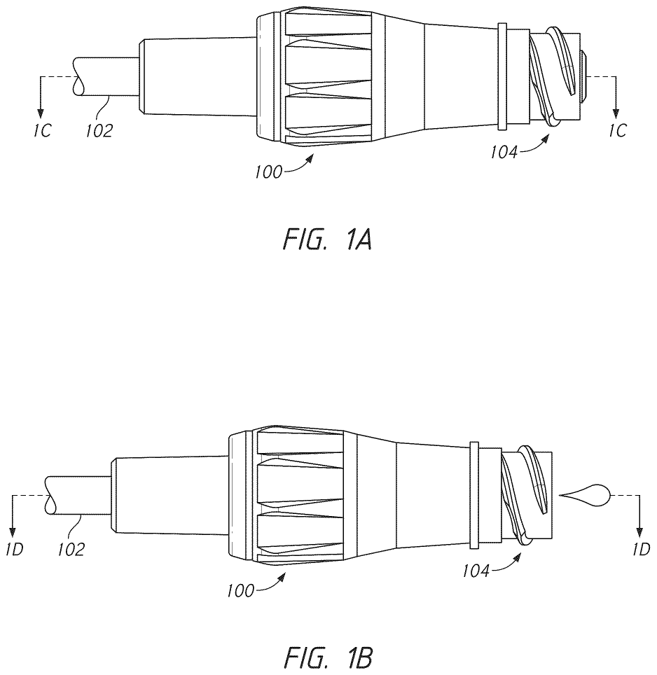

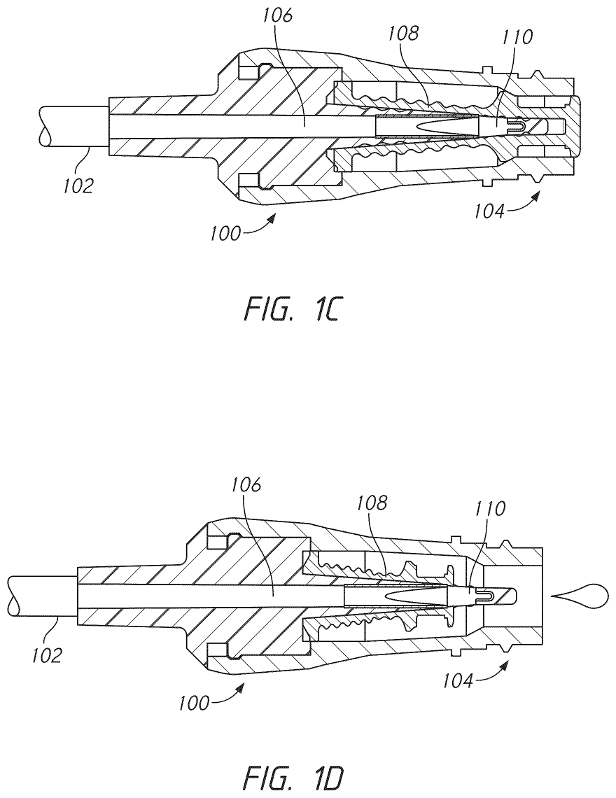

FIGS. 1A-1D illustrates an example of a connector 100 that can enable a medical professional to access a patient's vascular system (e.g., a circulatory system) through a fluid line, such as a tubing 102 and/or a catheter line. FIGS. 1A and 1B illustrate side views of the connector 100 when in a closed position and an opened position, respectively. FIGS. 1C and 1D illustrate side cross-sectional views of the connector 100 when in a closed position and an opened position taken along lines 1C-1C and 1D-1D of FIGS. 1A and 1B, respectively. In some embodiments, a catheter line can be inserted into a patient's arm. The catheter line can penetrate the skin of the arm and can be fluidly connected with the patient's bloodstream. The catheter line can be connected to a length of tubing 102 attached to a connector 100 and/or connected directly to the connector 100.

The connector 100 can be a fluid connector and/or a mechanical connector. The connector 100 can be configured to place the catheter line and/or tubing 102 in fluid communication with another medical implement (e.g., syringe or I.V. bag line). The connector 100 can mechanically couple the tubing 102 to the other medical implement. The connector 100 can include threads 104 configured to threadably couple with another portion of the complementary medical implement. In some embodiments, the connector 100 can be a needleless medical connector. In some embodiments, the connector 100 can be configured to accommodate any standard medical connector, such as ANSI (American National Standards Institute, Washington, D.C.) or other applicable standards. Some embodiments use a connector that is a MicroClave® neutral displacement connector commercially available from ICU Medical, Inc. Some embodiments use a connector that is a Clave® needle-free connector commercially available from ICU Medical, Inc. Various embodiments of a connector of this type are illustrated and described in U.S. Pat. No. 5,685,866, which is incorporated herein by reference in its entirety. The connector 100 described herein can be a needless medical connector including at least female and/or male luer portions. It is contemplated that many of the embodiments disclosed herein can be used with other types of connectors. Several embodiments use different types of connectors, including those that do not conform to recognized standards.

In some embodiments, the connector 100 may comprise a flow controller to selectively prevent and/or inhibit fluid from flowing through an internal passage of the connector 100 when the connector 100 is in a closed position (as shown in FIGS. 1A and 1C) and to selectively allow fluid to flow through the internal passage when the connector 100 is in an opened position (as shown in FIGS. 1B and 1D). Flow controllers can be formed by valves and/or seals that open and close passages. The flow controller can be and/or include a pump assembly, a valve assembly, a seal assembly, a plug assembly, and/or a system that pumps and/or selectively seals. In some embodiments, the flow controller is a valve that has an opened position to allow fluid to pass through the valve and a closed position that inhibits fluid from passing through the valve.

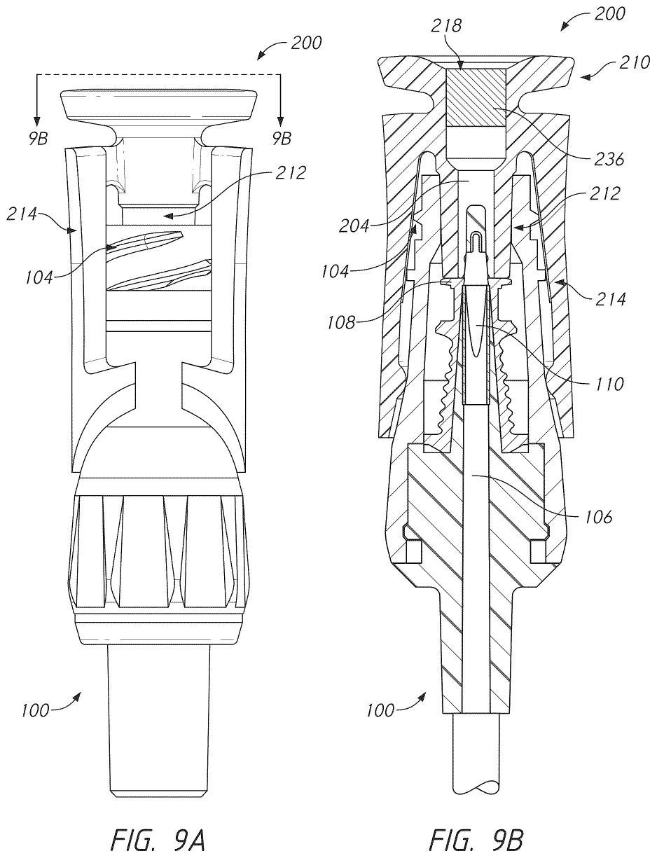

As illustrated in FIG. 1C, a seal 108 (e.g., a flow controller) can block, close, and/or seal an exit 110 (e.g., an aperture) from a passage 106 such that fluid is inhibited or prevented from moving past the seal 108 from the passage 106. Moving at least a portion of the seal 108 in a distal direction (as shown in FIG. 1D) can unblock and/or unseal the exit 110 such that fluid can move out of the passage 106 and past the seal 108. Mechanically coupling the connector 100 to a separate medical implement (e.g., as shown in FIGS. 9A and 9B discussed herein) can cause at least a portion the seal 108 to move distally relative to the exit 110 to establish fluid communication between the passage 106 and the separate medical implement. The passage 106 can be a fluid channel configured to be selectively opened (to allow fluid flow out of the connector 100) and closed (to block and/or inhibit fluid flow out of the connector 100). In some embodiments, one or more portions of the connector 100 may be transparent to permit a visual indication of when the connector 100 is primed and/or when a fluid (e.g., blood) enters the connector 100.

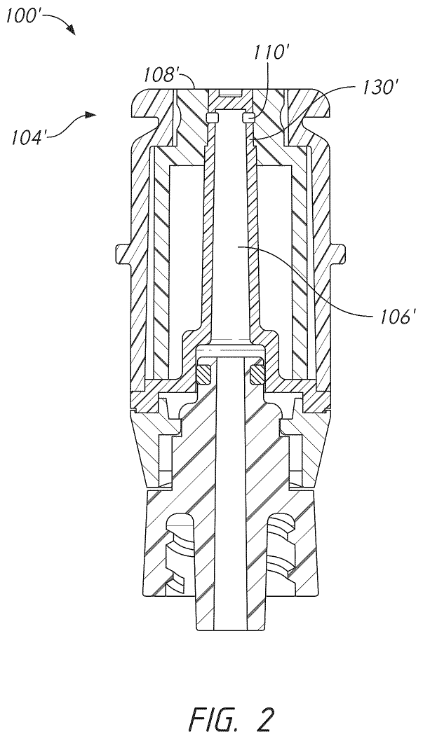

Although some embodiments disclose the use the connector 100 illustrated in FIGS. 1A-1D, it will be understood by one having skill in the art that other types of mechanical connectors, fluid connectors, and flow controllers may be utilized. In some embodiments, as described above, medical connectors for use in a vascular access system may not conform to recognized standards. For example, FIG. 2 illustrates a cross-sectional view of one embodiment of a medical connector 100′ that is not configured to conform to applicable connection standards. Such a connector is disclosed in greater detail in U.S. patent application Ser. No. 14/199,836, entitled “MEDICAL CONNECTORS WITH FLUID-RESISTANT MATING INTERFACES, filed on Mar. 6, 2014, now published as U.S. Publication No. 2014/0246616 A1, the entire disclosure of which is hereby incorporated by reference and made a part of the present specification. This can be helpful where it is desirable that the particular connector be used within the system. In some embodiments, a base of the medical connector may not conform to connection standards but the threads 104′ or other upper connection mechanism may conform to connection standards. In some embodiments, the threads 104′ or upper connection mechanism may not conform to connection standards, while the base does conform to such standards. In some embodiments, neither the upper connection mechanism nor the base conforms to applicable connection standards.

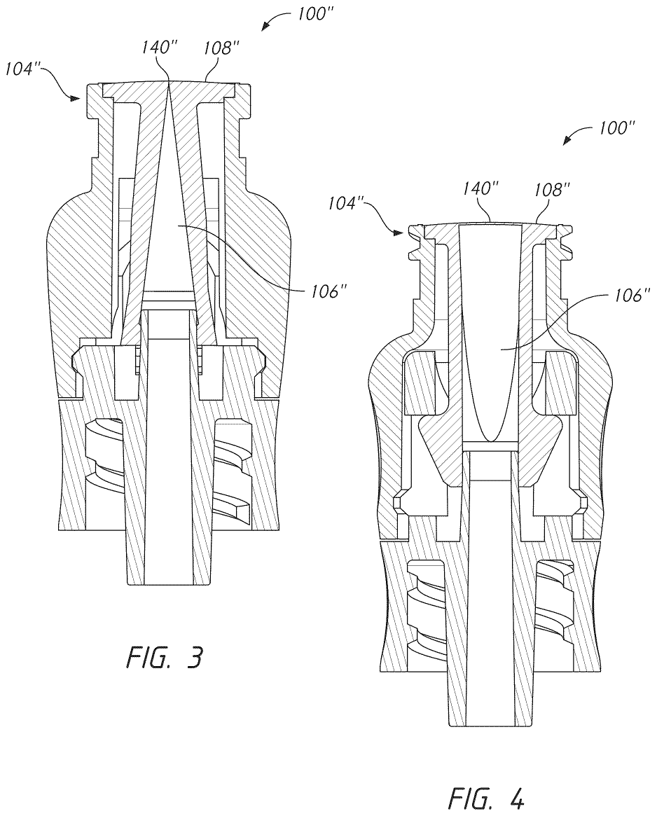

In some embodiments, a medical connector may have a fluid passage that is substantially defined by a movable flow controller and/or valve without an internal projection that extends through the flow controller. For example, FIGS. 3 and 4 illustrate one embodiment of a medical connector 100″ that includes a flow controller 108″ that accommodates a luer projection of a complementary medical connector to open the fluid passage 106″. Examples of medical connectors having the same or similar features as medical connector 100″ are further explained in International Patent Application Serial No. PCT/US2013/069312, entitled “MEDICAL CONNECTOR,” filed on Nov. 8, 2013, now published as International Patent Publication No. WO 2014/074929, the entire content of which is hereby incorporated by reference and made a part of the present specification.

Prior to insertion into a patient, some vascular access assemblies (e.g., a catheter line, tubing 102, and/or connector 100) may include fluid, such as gas and/or air. In some cases when the end of the tubing 102 that is not inserted into the patient is connected to a medical connector, such as connector 100 as illustrated in FIG. 1A, any air initially present within the tubing 102 upon insertion into the patient cannot escape the tubing 102 and may be transferred into the patient. If this air is allowed to enter the circulatory system (e.g., into the blood inside the patient), the fluid may result in gas embolic complications.

A catheter assembly and/or connector 100 may be “primed” by filling the catheter assembly and/or connector 100 with liquid and by removing any gas, such as air, from the catheter assembly. In some instances, a connector 100 may be manually moved into the opened position (shown in FIG. 1B) until all or a portion of the air has been purged through the connector 100 and the blood from the patient's vasculature fills the tubing 102 and the connector 100. With reference to FIGS. 1C and 1D, to remove the fluid (e.g., a gas) from the connector 100, blood from a patient's vasculature can flow into the tubing 102, then into the passage 106 located inside of the connector 100, and out of the connector 100 through the exit 110 when the connector 100 is in the opened position. The blood can then flow into another medical implement. This blood flow can remove any fluid (e.g., the air within the tubing 102 and/or connector 100) to “prime” the catheter assembly and/or connector 100.

In some instances, as soon as the tubing 102 and the connector 100 are properly primed, a health care provider can quickly activate the closing mechanism of the connector 100 to transition the connector 100 from the opened position to the closed position (as shown in FIGS. 1B and 1A, respectively) to rapidly stop the flow of fluid through the connector 100. However, it may be difficult to prime the connector 100 without exposing the surrounding environment to a patient's blood that may escape from the connector 100 when in the opened position and/or without forcing air into one or more medical implements to which the connector 100 may be attached.

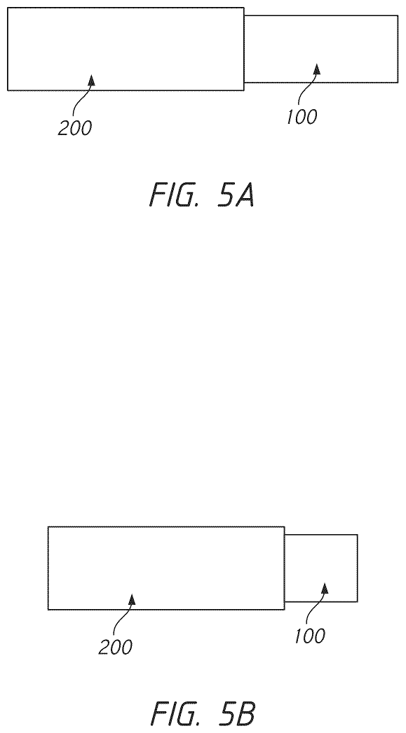

When catheter assemblies and/or connectors contain air, one or more caps can prime the catheter assembly and/or connector 100 to remove any unwanted air within a fluid line. With reference to FIGS. 5A and 5B, in such embodiments, a separate priming cap 200 can be attached to an end of the connector 100. The priming cap 200 can be structured in many different ways and can be advantageously implemented.

For example, a priming cap 200, as described herein, may be a single-use and/or disposable device. As described above, the priming cap 200 can be attached to an end of the connector 100. In some embodiments, as described in more detail below, the priming cap 200 can form a closed system and can allow air to be easily vented out of the connector 100 and/or tubing 102, which can be advantageous for several reasons. For example, the closed system can help to limit a patient's risk for bloodstream infection by limiting openings for bacterial entry and/or reducing the risk of occupational exposure. In some embodiments, the closed system can minimize the risk of touch-point contamination. In some embodiments, the closed system can help to reduce the amount of time required to prime the connector 100. While the priming cap 200 may be described in the context of priming connector 100, such as a needleless connector, the priming cap 200 can be implemented to prime other medical devices and instruments, such as a catheter insertion device, including an extension set, an IV line to or from an IV bag or patient, and/or the like.

The ability to vent air through the priming cap 200 can allow the priming cap 200 to be used in a self-priming extension set. As described in more detail below, the self-priming extension set may not require a fluid or other solution, such as saline or medication, to prevent air from entering the attached medical device. This can simplify the process of establishing an IV line and/or collecting a blood specimen. Such configurations can help to reduce the amount of time for an IV line to be primed and established. Thus, such configurations can help to reduce the risk of occupational exposure to blood.

FIG. 5A schematically illustrates an example of the priming cap 200 and the connector 100 in a first position (e.g. an inactivated position). FIG. 5B schematically illustrates an example of the priming cap 200 and the connector 100 in a second position (e.g., an activated position). As will be described in more detail below, according to some embodiments, the priming cap 200 can include a priming activation portion, such as a conduit housing, and an external member, such as a resilient member. In some embodiments, the priming cap 200 overlaps with at least a portion of the connector 100. For example, in some embodiments, the external member of the priming cap 200 can surround at least a portion of an exterior wall of the connector 100 and the activation portion can be positioned within and/or adjacent to at least a portion of the connector 100. In some embodiments, the external member may secure the priming cap 200 to the connector 100 such that the priming activation portion does not open a flow controller of the connector 100 to open fluid communication between the connector 100 and the priming cap 200 (e.g., the first, inactivated position). In some embodiments, to open the flow controller, the external member may remain secured to the connector 100. For example, the priming cap 200 can remain secured to at least a portion of the exterior of the connector 100 and the activation portion and/or the external member can slide into and/or about the connector 100 to open the flow controller of the connector 100 (e.g., the second, activated position). Such configurations can establish fluid communication between the connector 100 and the priming cap 200.

In some embodiments, as described herein, all or a portion of the priming cap 200 can be rigid. In some embodiments, all or a portion of the priming cap 200 can be flexible and/or resilient. For example, in some embodiments in which all or a portion of the priming cap 200 is flexible, the priming cap 200 may engage the connector 100 in a friction fit, such as a screw-less friction fit. In some embodiments, when the priming cap 200 slides into and/or about the connector 100 (e.g., the second, activated position), at least a portion of the priming cap 200 can expand outwardly. For example, the external member can expand and be secured to the priming cap 200 as the priming cap 200 is pushed into the connector 100. In some configurations, the priming activation portion may additionally, or alternatively, slide into the connector 100 and frictionally engage an interior wall of the connector 100 and/or the flow controller (e.g., seal 108″ as shown in FIGS. 3 and 4) or fluid passage (e.g., passage 106″ as shown in FIGS. 3 and 4) to secure the priming cap 200 to the connector 100. Accordingly, in some embodiments, the priming cap 200 is frictionally secured to the connector 100.

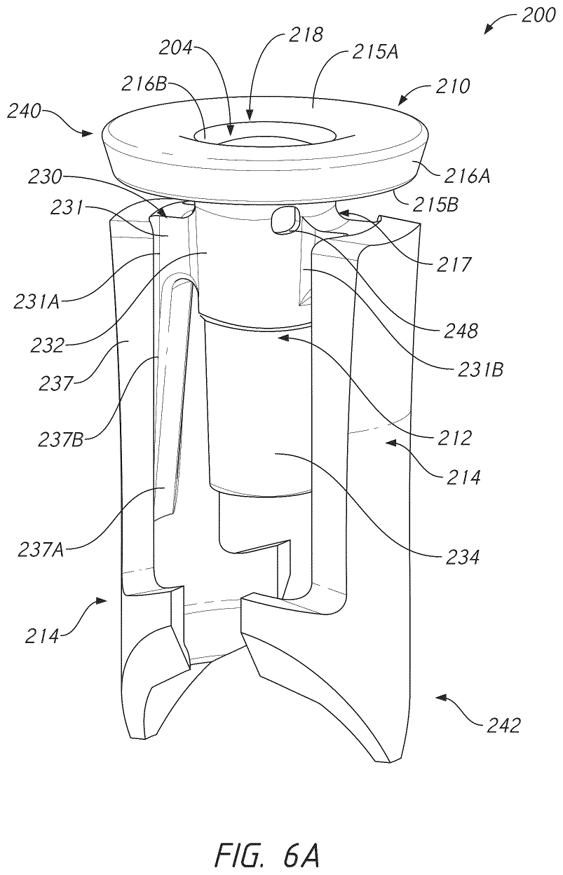

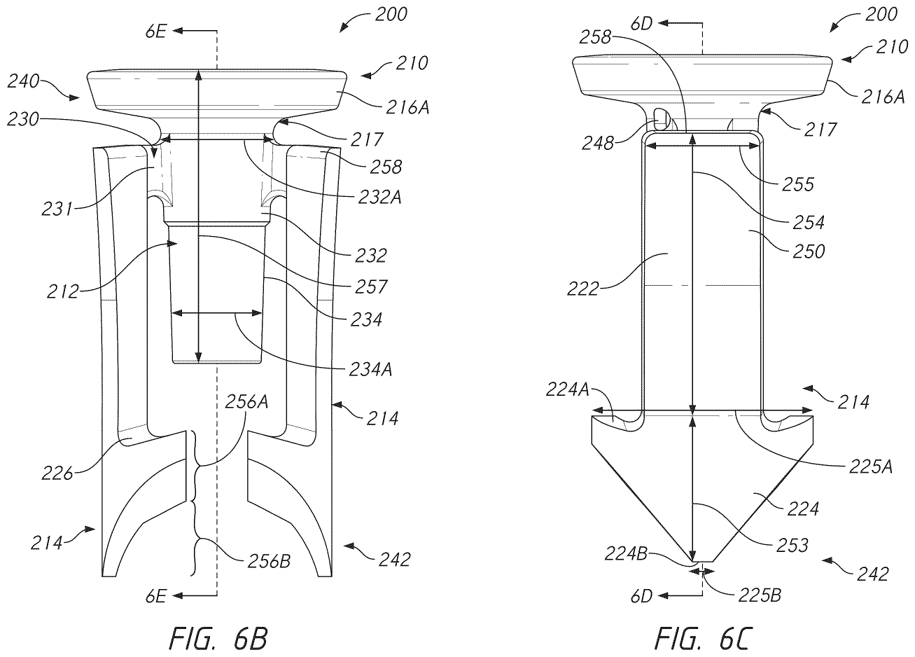

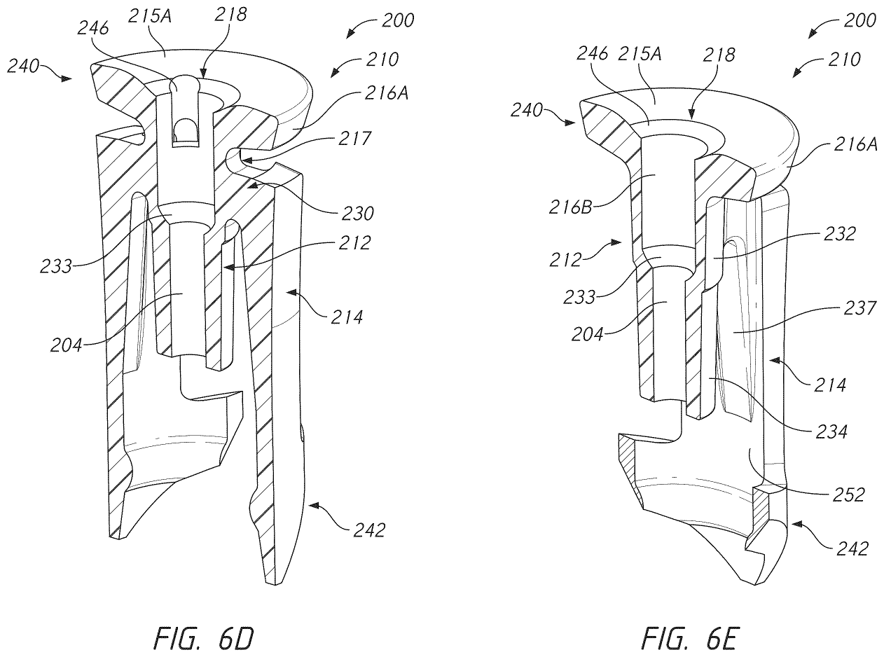

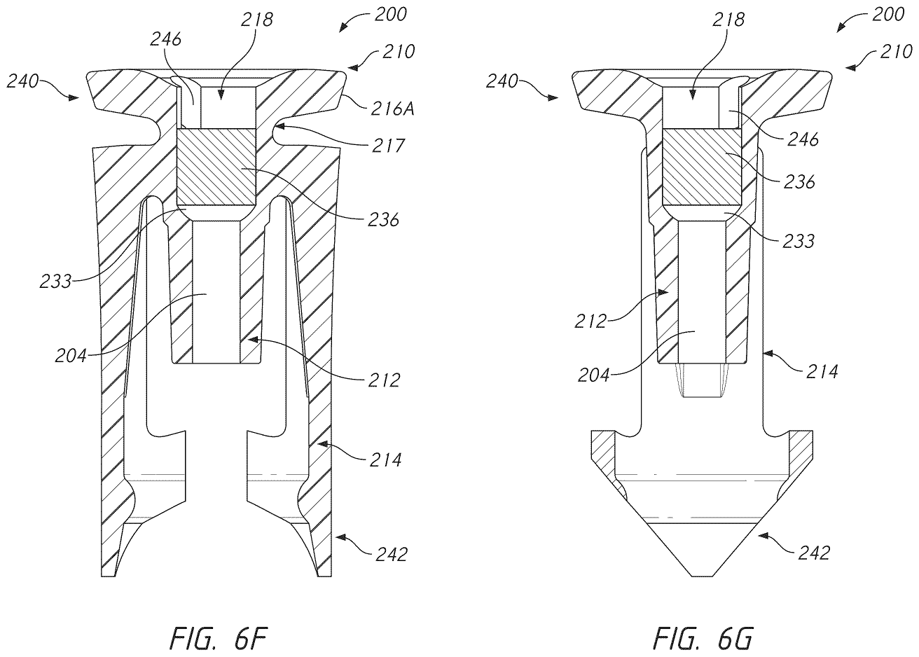

FIGS. 6A-6G provide an example of the priming cap 200 that can be used with the connector 100, such as a female connector. In particular, FIG. 6A is a front perspective view of a priming cap 200, FIGS. 6B-6E are front, side, front perspective cross-sectional, and side perspective cross-sectional views of the priming cap 200 of FIG. 6A, respectively, and FIGS. 6F and 6G are front cross-sectional and side cross-sectional views of the priming cap 200 of FIG. 6A including a plug 236. Unless otherwise noted, reference numerals in FIGS. 6A-6G refer to components that are the same as or generally similar to the components in the remaining figures and/or embodiments discussed herein. It will be understood that the priming cap 200 shown in FIGS. 6A-6G can be used with any of the embodiments described and/or contemplated herein. It will also be understood that any of the embodiments described and/or contemplated herein can be modified to be used with the priming cap 200 shown in FIGS. 6A-6G. As with all embodiments in this specification, any feature, structure, material, method, or step that is described and/or illustrated in the embodiment of FIGS. 6A-6G can be used with or instead of any feature, structure, material, method, or step that is described and/or illustrated in any other embodiment of this specification.

In some embodiments, the priming cap 200 can include a structure to transition the connector 100 between the closed and opened positions. For example, the structure can include a conduit housing 212 configured to push against at least a portion (e.g., seal 108 or an end) of the connector 100. The conduit housing 212 can include an internal conduit 204. The internal conduit 204 can define a fluid passageway that passes through at least a portion of the conduit housing 212. When at least a portion of the conduit housing 212 pushes against at least a portion of the connector 100 (e.g., seal 108), the connector 100 may transition to the opened position such that fluid is permitted to escape from inside of the connector 100 through the exit 110 (as shown in FIG. 1D). The fluid can pass from the connector 100 and through at least a portion of the fluid passageway defined by the internal conduit 204. As described herein, the fluid passageway can lead to an exit bore 218 of the priming cap 200.

In some configurations, at least a portion of the priming cap 200 and/or the connector 100 is clear and/or transparent. Such configurations can desirably allow a health care provider to visualize the flow path of fluid (e.g., blood) through the connector 100 and/or the priming cap 200. This can allow the health care provider to ensure that the catheter line, IV line, connector, and/or extension set has been properly primed and/or established. Such configurations can allow the health care provider to visualize the connector 100 flushing after blood draws and/or administration of certain medications.

As shown in FIGS. 6A-6G, the priming cap 200 can include an upper portion 210, the conduit housing 212, and one or more resilient arms 214. In some embodiments, at least one of the upper portion 210, the conduit housing 212, and/or the resilient arms 214 are flexible. In some embodiments, at least one of the upper portion 210, the conduit housing 212, and/or the resilient arms 214 are substantially rigid. The upper portion 210 can be positioned at a first end 240 of the priming cap 200. The first end 240 can be positioned opposite a second end 242 of the priming cap 200. The second end 242 of the priming cap 200 can be positioned adjacent to at least a portion of the connector 100 when assembled.

The upper portion 210 can be shaped and configured to substantially permit air to pass through and/or exit, while preventing liquid from passing through and/or exiting, the first end 240 of the priming cap 200 through the exit bore 218. For example, in some embodiments, the upper portion 210 may comprise a filter and/or plug 236 (as shown in FIGS. 6F and 6G and described herein). The upper portion 210 can have a circular cross-sectional shape. In some embodiments, the upper portion 210 can have a trapezoidal, square, rectangular, and/or oval cross-sectional shape. As shown in the illustrated embodiment, the upper portion 240 can have an upper wall 215A, a lower wall 215B, an outer side wall 216A, and an inner side wall 216B. The outer side wall 216A can extend between the upper wall 215A and the lower wall 215B. The outer side wall 216A can be integrally formed with the upper wall 215A and/or the lower wall 215B.

In some embodiments, the upper wall 215A and/or the lower wall 215B can be substantially flat. In some embodiments, the outer side wall 216A includes a tapered profile. For example, the upper wall 215A can include an outer diameter and the lower wall 215B can include an outer diameter. In some embodiments, the outer diameter of the upper wall 215A can be greater than the outer diameter of the lower wall 215B. In some embodiments, the outer diameter of the upper wall 215A is approximately equal to or less than the outer diameter of the lower wall 215B. The tapered profile of the outer side wall 216A can help to reduce the overall bulkiness of the priming cap 200. In some configurations, the tapered profile of the outer side wall 216A provides a better fit in the health care provider's hands and can allow the priming cap 200 to be more easily used.

The upper portion 210 can be connected to the conduit housing 212. In some embodiments, the upper portion 210 is integrally formed with a first end of the conduit housing 212. As shown in at least FIGS. 6A-6D, the upper portion 210 transitions smoothly between the lower wall 215B and the conduit housing 212 at a transition region 217. The transition region 217 can define an annular groove. The annular groove can interact with certain features of other medical devices to axially restrain movement of the priming cap 200.

The conduit housing 212 can include an upper housing 232 and a lower housing 234. The upper housing 232 and the lower housing 234 can be integrally formed. In some embodiments, the lower housing 234 can be configured to contact at least a portion (e.g., seal 108) of an interior of the connector 100. When assembled, the lower housing 234 can establish a fluid flow path between the connector 100 and the priming cap 200. Thus, the corresponding conduit housing 212 can define an activation member that can transition the connector 100 between the opened position and the closed position. For example, the lower housing 234 can be shaped and sized to fit within at least a portion of the connector 100 and, when activated, the lower housing 234 can push against a portion (e.g., seal 108) of the connector 100 to permit fluid to escape from the connector 100.

In some embodiments, the lower housing 234 can be substantially cylindrical. In some embodiments, the lower housing 234 is tapered. For example, an outer surface of the lower housing 234 can be tapered away from the upper housing 232 to correspond to a luer taper of the connector 100.

With reference to FIG. 6B, in some embodiments, the lower housing 234 has a maximum outer diameter 234A and the upper housing 232 has a maximum outer diameter 232A. The maximum outer diameter 234A of the lower housing 234 can be less than the maximum outer diameter 232A of the upper housing 232. In some embodiments, the maximum outer diameter 234A of the lower housing 234 can be equal to or greater than the maximum outer diameter 232A of the upper housing 232. In some embodiments, an interior volume of the lower housing 232 is less than an interior volume of the upper housing 234. For example, the lower housing 234 can be positioned inwardly relative to the upper housing 232.

As shown in FIGS. 6D and 6E, a transition region within the internal conduit 204 between the upper housing 232 and the lower housing 234 can form an internal ledge 233. The internal ledge 233 can be tapered. In some embodiments, the internal ledge 233 is tapered inwardly from the upper housing 232 towards the lower housing 234. In some embodiments, the internal ledge 233 forms a stepped configuration such that the internal ledge 233 is substantially flat.

In some embodiments, the priming cap 200 can include a plug 236 (as shown in FIGS. 6F and 6G) that is configured to permit air to pass through the plug 236 but inhibit the passage of liquid through the plug 236. For example, when the priming cap 200 and the connector 100 are in the second, activated position (e.g. when a portion of the priming cap 200 is pressed into fluid communication with the connector 100) and a fluid passageway is established between the connector 100 and the priming cap 200, fluid, such as the patient's blood, may be permitted to flow between the connector 100 and the priming cap 200. The plug 236 can allow air to be vented from the tubing 102 and/or connector 100 and exit the priming cap 200 by passing through the plug 236, while preventing any liquid from the tubing 102 and/or connector 100 from exiting the priming cap 200. In some embodiments, the plug 236 includes a filter, filter membrane, and/or a hydrophobic material, among other materials. Such configurations can allow the catheter line, the tubing 102, the connector 100, IV line, and/or other medical device, to be properly primed and to easily remove air without the need to introduce additional fluids, such as saline or other medications, to remove air. Such configurations can be desirably safer to use and can help to limit infections.

With continued reference to FIGS. 6F and 6G, in some embodiments, the plug 236 is positioned within an interior volume of at least a portion of the conduit housing 212. For example, the plug 236 can be positioned within and/or substantially fill an internal cavity located within at least the upper housing 232. Such configurations can allow fluid to pass through the internal conduit 204 of the lower housing 234, but substantially limit or prevent liquid from passing through and/or exiting out of the internal conduit 204 of the upper housing 232 through the exit bore 218. In some embodiments, the plug 236 can be positioned within and/or substantially fill at least the lower housing 234. In some embodiments, the plug 236 can be positioned within and/or substantially fill all or a portion of the upper and/or lower housings 232, 234. Thus, the upper housing 232 and/or the lower housing 234 can be shaped and sized to receive the plug 236. In some embodiments, the upper housing 232 and/or the lower housing 234 is substantially cylindrical. As shown, the internal ledge 233 can be shaped to retain the plug 236 within the upper housing 232. For example, the internal ledge 233 provides a platform for the plug 236 to sit within the upper housing 232. The internal ledge 233 may prevent all or a portion of the plug 236 from sliding into another portion of the conduit housing 212, such as the lower housing 234.

As shown in FIG. 6D, the priming cap 200 can include a venting notch 246. The venting notch 246 can extend along an inner side wall of at least a portion of the conduit housing 212 and/or the upper portion 210. For example, the venting notch 246 can extend along the inner side wall 216B of at least a portion of the upper housing 232. The venting notch 246 can be cut into a side wall of at least a portion of the upper housing 232 and the upper portion 210. In some embodiments, at least a portion of the venting notch 246 extends through the side wall of the conduit housing 212 and/or the upper portion 210. For example, the venting notch 246 can include a venting hole 248 (as shown in FIGS. 6A and 6C). The venting hole 248 can be positioned at a lower region of the venting notch 246 and/or an upper region of the upper housing 232. The venting notch 246 and/or the venting hole 248 can enhance the venting properties of the plug 236, among other portions of the priming cap 200. The venting notch 246 and/or the venting hole 248 can further allow trapped air to escape through the priming cap 200 when the connector 100 is primed. For example, as a health care provider transitions the priming cap 200 and the connector 100 from the first, inactivated position towards the second, activated position (as described herein), at least a portion of the provider's hand (e.g., a thumb) may at least partially cover the exit bore 218. In such instances, the venting notch 246 and the venting hole 248 may provide a method to facilitate air from escaping the priming cap 200, even if a substantial portion of the exit bore 218 is being covered. Such configurations can help to ensure that the connector 100 is properly primed and that air is not pushed through an opposite end of the connector 100 and/or the tubing 102.

In some embodiments, the conduit housing 212 can be connected to one or more external members (as described herein) in the form of the resilient arms 214. In some embodiments, the priming cap 200 may not comprise any external member and/or resilient member 214. The conduit housing 24 of the priming cap 200 may be configured to maintain engagement between the priming cap 200 and the connector 100 via any suitable method (e.g., friction fit or screw fit) without the requirement of any additional connection members.

As shown in FIGS. 6A-6G, the conduit housing 212 can be connected to the resilient arms 214 by a connecting member 230. The connecting member 230 can extend from at least a portion of the conduit housing 212 to at least a portion of the resilient arms 214. The connection member 230 can be laterally centered with respect to a lateral width of the resilient arms 214. In some embodiments, the connection member 230 is positioned offset from a lateral center of the resilient arms 214. For example, the connection member 230 can be positioned closer to one lateral side of the resilient arms 214 than another lateral side.

The connecting member 230 can include a lateral connection portion 231 and a vertical connection portion 237. The lateral connection portion 231 can have an inner side 231A and an outer side 231B. The inner side 231A can be coupled with at least a portion of the upper housing 232 and the outer side 231B can be coupled with at least a portion of the resilient arms 214. For example, the inner side 231A can extend along at least ⅔ of a length of the upper housing 232. In some embodiments, the inner side of the lateral connection portion 231 extends along at least ½ a length of the upper housing 232.

In some embodiments, the vertical connection portion 237 includes an inner side 237A and an outer side 237B. The inner side 237A can include a tapered surface. The tapered surface can extend from a bottom surface of the lateral connection portion 231 towards an inner side wall of the resilient arms 214 and/or away from an outer wall of the conduit housing 212. For example, the tapered surface of the inner side 237A can be tapered downwardly from the lateral connection portion 231 to the inner side wall of the resilient arms 214. In some embodiments, the inner side 237A extends downwardly along the inner side wall of the resilient arms 214 to a position lower than a bottom surface of the conduit housing 212. The tapered surface can help to secure the priming cap 200 within the connector 100 when assembled. For example, when the priming cap 200 activated and/or is lowered towards the connector 100 and the conduit housing 212 contacts the connector 100, at least a portion of the connector 100 can slide between the tapered surface of the inner side 237A and an outer wall of the conduit housing 212. As the priming cap 200 continues to be pushed into the connector 100, at least a portion of the connector 100 can be secured within the space between the tapered surface of the inner side 237A and the outer wall of the conduit housing 212 by, for example, a friction fit among other arrangements. In some embodiments, the tapered surface of the inner side 237A allows at least a portion of the connector 100 to be wedged between the tapered surface of the inner side 237A and at least a portion of the conduit housing 212.

As discussed above, the priming cap 200 can include resilient arms 214. The priming cap 200 can include one, two, three, or four or more resilient arms 214. In some instances in which the priming cap 200 comprises two or more resilient arms 214, the resilient arms 214 may be spaced at equal distances along an outer circumference of the priming cap 200. For example, in an embodiment of the priming cap 200 that includes two resilient arms 214, the resilient arms 214 may be located at diametrically opposed sides of the priming cap 200. In some embodiments, two or more resilient arms 214 may define and/or at least partially surround an interior space. The interior space may be configured to at least partially receive at least a portion of the connector 100. The resilient arms 214 can be supported by the connection portions 230 and spaced outwardly away from the conduit housing 212. The resilient arms 214 can be shaped to define an indicator. For example, the shape of the resilient arms 214 can indicate a direction that the priming cap 200 should be pushed to engage with the connector 100. In some embodiments, the shape of the resilient arms 214 indicates a side of the priming cap 200 that should be secured to the connector 100. As shown in FIG. 6C, for example, the resilient arms 214 are shaped as an arrow.

With continued reference to FIG. 6C, in some embodiments, the resilient arms 214 include a base 222 and a head 224. The base 222 and the head 224 can be integrally formed. The base 222 and the head 224 can form the shape of an arrow indicator. For example, the base 222 can be substantially rectangular and the head 224 can be substantially triangular and/or trapezoidal. As shown, the head 224 can include an upper head region 224A and a lower head region 224B. The lower head region 224B can have a lateral length 225B and the upper head region 224A can have a lateral length 225A. The lateral length 225B of the lower head region 224B can be less than the lateral length 225A of the upper head region 224A such that the triangular- and/or trapezoidal-shaped head 224 is pointing in a downward direction. The shape of the resilient arms 214 can quickly indicate to the health care provider the direction that the priming cap 200 should be oriented with respect to the connector 100.

The resilient arms 214 can include an outer surface 250 (as shown in FIG. 6C) and an inner surface 252 (as shown in FIG. 6E). In some embodiments, the inner surface 252 and/or the outer surface 250 of the resilient arms 214 can be rounded. For example, the inner surface 252 can have an inner diameter and form at least a portion of an inner circumference. The outer surface 250 can have an outer diameter and form at least a portion of an outer circumference. The inner circumference of the inner surface 252 and the outer circumference of the outer surface 250 can be concentric with the inner conduit 204. In some embodiments, the outer surface 250 and the inner surface 252 are shaped to correspond to a shape of the connector 100. For example, the inner surface 252 of the resilient arms can be shaped to define an interior volume configured to receive at least a portion of the connector 100.

In some embodiments, the head 224 can have a vertical length 253 that is approximately one-half a vertical length 254 of the base 222. In some embodiments, the head 224 can have a vertical length 253 that is approximately ⅛, ¼, ⅓, ⅔, and/or ¾ or greater than the vertical length 254 of the base 222. In some embodiments, the vertical length 253 is greater than or less than one-half the vertical length 254. In some embodiments, the vertical length 253 is greater than or less than ⅛, ¼, ⅓, ⅔, and/or ¾ or greater the vertical length 254. In some embodiments, the vertical length 254 of the base 222 is approximately equal to a length 257 (as shown in FIG. 6B) between a top surface of the upper portion 210 and a bottom surface of the lower housing 234 of the conduit housing 212.

As shown in at least FIG. 6C, in some embodiments, an outer width 255 of the base 222 is approximately equal to or larger than a diameter of the upper housing 232 of the conduit housing 212. In some embodiments, the lateral length 225A of the upper head region 224B is a maximum outer width of the head 224. In some embodiments, the length 225A of the head 224 is less than a diameter of the bottom wall of the upper portion 210. In some embodiments, the lateral length 225A is less than a diameter of the top wall of the upper portion 210. In some embodiments, the outer width 255 of the base 222 is approximately one-half the lateral length 225A of the head 224. In some embodiments, the outer width 255 of the base 222 is approximately ⅛, ¼, ⅓, ⅔, and/or ¾ or greater the lateral length 225A of the head 224.

In some embodiments, at least a portion of the outer surface 250 of the resilient arms 214 is tapered and/or concave. For example, a top outer edge 258 (as shown in FIGS. 6B and 6C) of the outer surface 250 of the resilient arms 214 can be positioned outwardly a greater distance relative to the conduit housing 212 than a bottom outer edge 259 of the outer surface 250. In some embodiments, at least a portion of the outer surface 250 is contoured. For example, the outer surface 250 can include a thicker or wider portion near a top region of the resilient arms 214 and a thinner or narrower portion near a central region or a lower region of resilient arms 214. The shapes described herein can provide tactile confirmation of the proper placement of a user's fingers on the priming cap 200 during use and/or provide a more comfortable gripping surface. In some embodiments, an outward projection or projections (not shown) can be incorporated on the resilient arms 214 to provide additional or more effective gripping surfaces on the priming cap 200. In some configurations the shapes of the resilient arms 214 described herein can desirably help to secure the priming cap 200 to the connector 100. Such configurations can help to maintain an engagement between at least a portion of the priming cap 200 and at least a portion of the connector 100.

In some embodiments, the resilient arms 214 include a retaining structure, such as a bumper 226 (as shown in FIG. 6B). In some embodiments, the bumper 226 is expandable. In some embodiments, the bumper 226 can help to secure the priming cap 200 to the connector 100. For example, bumper 226 can secure the priming cap 200 to at least a portion of the connector 100 (e.g., threads 104). In some configurations, the bumper 226 can be configured to be retained within a corresponding groove and/or notch, such as a screw thread 104 of the connector 100. For example, the bumper 226 can engage with a corresponding engagement feature of the connector 100, such as by a snap-fit arrangement and/or friction-fit arrangement, among others. The bumper 226 can secure the priming cap 200 to the connector 100 without activating the priming cap 200. Thus, the priming cap 200 and/or the connector 100 can be assembled in the first, inactivated position before use (e.g., during shipment and/or packaging or before priming).

In some configurations, when priming the connector 100, for example, the health care provider may only need to activate the priming cap 200 by pressing down on the previously assembled priming cap 200 into the connector 100. Thus, the priming cap 200 can be implemented in a self-priming and/or closed-system. Such configurations can help to save time since the priming cap 200 may already be secured to a portion of the connector 100 in the correct orientation.

The bumper 226 can be positioned along a portion of the inner surface 252 of the resilient arms 214, such as a portion of the head 224. In some embodiments, a lateral center of the bumper 226 is approximately aligned with a lateral center of the head 224. In some embodiments, with reference to FIG. 6B, the head 224 includes a region of increased thickness 256A and a region of decreased thickness 256B. In some embodiments, the bumper 226 is positioned along the region of increased thickness 256A. In some embodiments, a height of the bumper 226 is approximately one-half a maximum height of the region of increased thickness 256A. In some embodiments, a height of the bumper 226 is approximately ⅛, ¼, ⅓, ⅔, and/or ¾ or greater a maximum height of the region of increased thickness 256A. In some embodiments, the bumper 226 is positioned adjacent a lower end of the region of increased thickness 256A. In some embodiments, the bumper 226 is positioned adjacent the region of decreased thickness 256B. In some embodiments, the region of decreased thickness 256B forms a partial spherical shape configured to surround a portion of the connector 100.

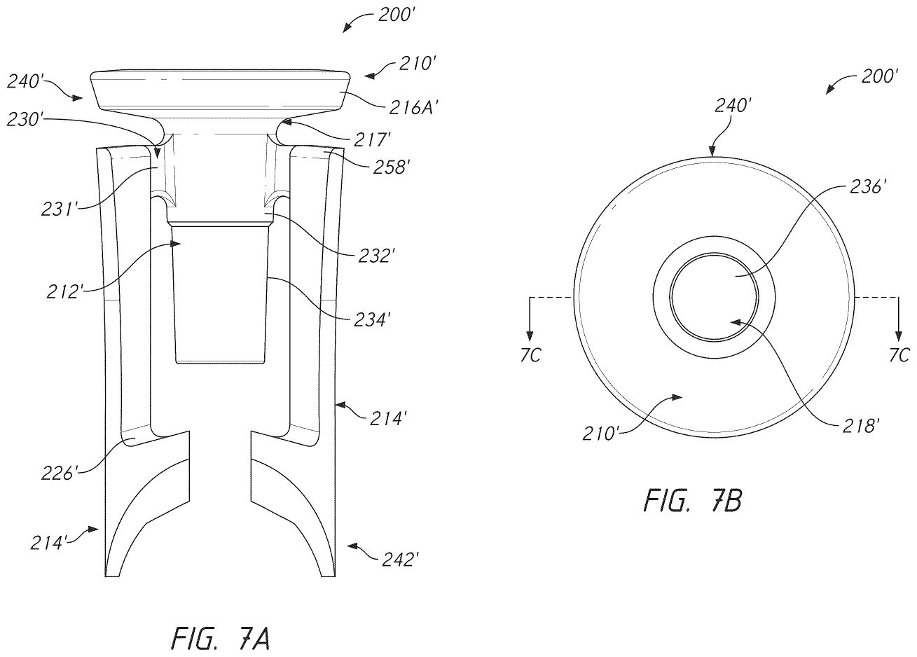

FIGS. 7A-7C are various views of a priming cap 200′, according to some embodiments. In particular, FIG. 7A is a front view of a priming cap 200′, and FIGS. 7B and 7C are top and front cross-sectional views of the priming cap 200′ of FIG. 7A, respectively. Unless otherwise noted, the priming cap 200′ as shown in FIG. 7A may include components that are the same as or generally similar to the components in the remaining figures and/or embodiments discussed herein. It will be understood that the priming cap 200′ shown in FIG. 7A can be used with any of the embodiments described and/or contemplated herein. It will also be understood that any of the embodiments described and/or contemplated herein can be modified to be used with the priming cap 200′ shown in FIG. 7A. As with all embodiments in this specification, any feature, structure, material, method, or step that is described and/or illustrated in the embodiment of FIGS. 7A-7C can be used with or instead of any feature, structure, material, method, or step that is described and/or illustrated in any other embodiment of this specification.

The internal conduit 204′ can receive and/or house any suitable filter and/or plug 236′ that permits the passage of liquid through the plug 236′, while inhibiting the passage of gas through the plug 236′, via any method discussed herein. In some embodiments, the priming cap 200′ may not include venting hole and/or venting notch to facilitate the functioning of the filter and/or plug 236′. For example, as the filter and/or plug 236′ is inserted into and/or placed within the internal conduit 204′ of the priming cap 200′ (e.g., through the exit bore 218′), insertion and proper placement of the plug 236′ may be facilitated by the smooth walls of the exit bore 218′ and/or internal conduit 204′. In some instances, an asymmetrical exit bore 218- and/or internal conduit 204′ may damage the plug 236′ and/or cause the plug 236′ to not seat properly. In some embodiments, not including the venting notch and/or the venting hole can advantageously enhance the venting properties of the plug 236′.

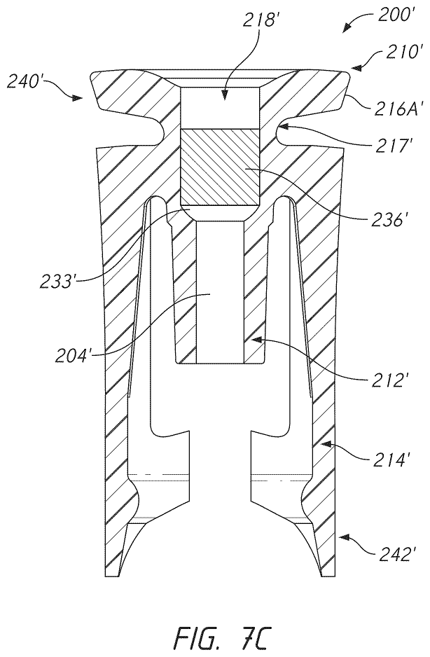

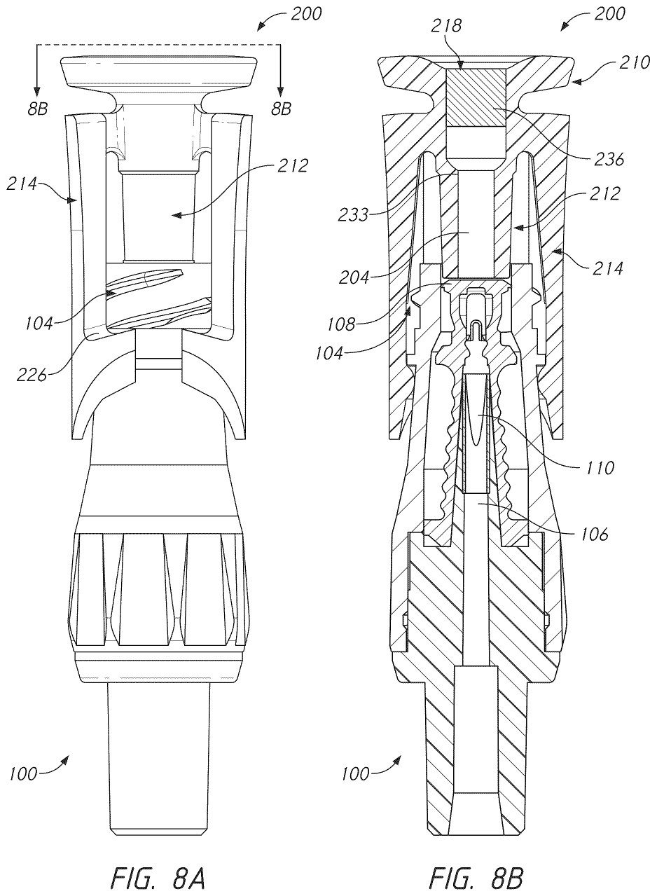

FIGS. 8A and 8B illustrate an embodiment of the priming cap 200 engaged with the connector 100 when in the first, inactivated position. In particular, FIG. 8A is a front view of a priming cap 200 engaged with the connector 100, and FIG. 8B is a front cross-sectional view of the priming cap 200 and connector 100 of FIG. 8A. Similarly, FIGS. 9A and 9B illustrate an embodiment of the priming cap 200 engaged with the connector 100 when in the second, activated position. In particular, FIG. 9A is a front view of a priming cap 200 engaged with the connector 100, and FIG. 9B is a front cross-sectional view of the priming cap 200 and connector 100 of FIG. 9A. Unless otherwise noted, it will be understood that any of the embodiments described and/or contemplated herein can perform any step and/or function discussed with reference to FIGS. 8A-9B. As with all embodiments in this specification, any feature, structure, material, method, or step that is described and/or illustrated in the embodiment of FIGS. 8A-9B can be used with or instead of any feature, structure, material, method, or step that is described and/or illustrated in any other embodiment of this specification.

With reference to FIGS. 8A and 8B, the figures illustrate a priming cap 200 coupled to a connector 100 in a first, inactivated position. As discussed herein, in some embodiments, the priming cap 200 can be coupled to the connector 100 in a first position such that the priming cap 200 does not open a flow controller of the connector 100 to open fluid communication between the connector 100 and the priming cap 200. When in the first position, as illustrated, the seal 108 of the connector 100 can seal the exit 110 such that the passage 106 is not in fluid communication with the internal conduit 204 of the priming cap 200.

As illustrated in the figures and described herein, the resilient arms 214 may facilitate to secure the priming cap 200 to the connector 100. In some embodiments, as shown in FIGS. 8A and 8B, the resilient arms 214 contact, cover, and/or overlay against or extend across at least a portion of an exterior surface of the connector 100, such as a proximal end region of the connector 100. In the illustrated embodiments, the resilient arms 214 facilitate coupling between the priming cap 200 and the connector 100. For example, as shown, the bumper 226 of the resilient arms 214 may be engaged with at least a portion of the connector 104 (e.g., a corresponding groove and/or thread 104) and at least a portion of the lower housing 234 of the conduit housing 212 is positioned within and/or adjacent to an end of the connector 100. The resilient arms 214 and/or bumper 226 can secure the priming cap 200 to the connector 100 when in this first position without activating the priming cap 200 and/or connector 100. Thus, the priming cap 200 and/or the connector 100 can be assembled in the first, inactivated position before use (e.g., during shipment and/or packaging, or before priming).

In some embodiments, as shown in FIG. 8B, when the priming cap 200 is coupled to the connector 100, a distal end of the conduit housing 212 of the priming cap 200 can be at least partially received by an opening of the connector 100. In some embodiments, during or after coupling of the priming cap 200 and the connector 100, the distal end of the conduit housing 212 of the priming cap 200 is moved adjacent to or into contact with at least the seal 108 of the connector. As illustrated, when the priming cap 200 is in the first position, the conduit housing 212 and the seal 108 are positioned in a sufficiently proximal direction from the exit 110 such that the connector 100 is maintained in the closed position and the seal 108 prevents fluid communication between the internal conduit 204 of the priming cap 200 and the passage 106 of the connector 100.

As illustrated in FIGS. 9A and 9B, in some embodiments, when the priming cap 200 and the connector 100 are transitioned to the second position form the first position, the conduit housing 212, and consequently the seal 108 of the connector 100, can be moved in a distal direction relative to the exit 110 of the connector 100. FIG. 9B shows that has the priming cap 200 is moved in a distal direction relative to the connector 100, the conduit housing 212 can force the seal 108 to move distally to transition the connector 100 into an opened position (e.g., as shown in FIGS. 1B and 1D). In some embodiments, the conduit housing 212 can compress the seal 108. In such an instance, when the connector 100 is in the opened position, there is no structure or material between the exit 110 and/or passage 106 of the connector 100 and the internal conduit 204 of the priming cap 200. The securing of the priming cap 200 and the connector 100 is this position permits any fluid located within the passage 106 of the connector 100 to flow into the internal conduit 204 within the priming cap 200. As discussed herein, as fluid flows into the internal conduit 204, the plug 236 of the priming cap 200 may permit any gas that flows into the internal conduit 204 to pass through the plug 236 and exit the priming cap 200 through the exit bore 218. However, as liquid flows into the priming cap 200, the plug 236 can inhibit the flow of the liquid through the plug 236 and out of the exit bore 218. Accordingly, the priming cap 200 may function to vent the connector 100 and/or priming cap 200 of any gas while priming the connector 100 and/or any connected structure (e.g., tubing 102) such that the connector 100 and/or any connected structure may become substantially devoid of gas. In some instances, a health care provider may wish to remove the priming cap 200 after the connector 100 and/or any connected structure has been sufficiently primed. In such an instance, as the health care provider disengages the priming cap 200 from the connector 100, it will be appreciated that the connector 100 would return to the initial, closed position and prevent any further fluid from flowing into and/or out of the connector 100.

In some embodiments, needleless connectors can be used that contain a proximal protrusion and passage that extends further into the seal than the connector 100 as illustrated in FIGS. 8A and 8B when the priming cap 200 and the connector are in the first position. The connector 100′ of FIG. 2 provides an example of one such embodiment. FIG. 2 illustrates a cross-sectional view of the medical connector 100′. As shown, in some embodiments the proximal protrusion 130′ can extend through the seal 108′ to a proximal face of the seal 108′ when the connector 100′ is in the first position. When the seal 108′ is moved distally into the opened position, it can expose the exit 110′ and place the passage 160′ into fluid communication with the priming cap 200.

In some embodiments, instead of having a proximal protrusion that defines an internal conduit, such as the passage, the seal can at least partially define the passage. The medical connector 100″ of FIGS. 3 and 4 provides an example of one such embodiment. FIG. 3 illustrates a cross-sectional view of the connector 100″, and FIG. 4 illustrates a cross-sectional view of the connector 100″ rotated 90 degrees. As illustrated, the seal 108″ can define a substantial portion of the passage 106″.

In some embodiments, for example as further shown by the connector 100″, a seal 108″ can be configured to remain in the same position when the connector 100″ is in the first position and when the connector 100″ is in the second position. When the connector 100″ moves to the second position, instead of having the conduit housing 212 of the priming cap 200 move the seal 108″ distally, the conduit housing 212 can pass through a slit 140″ of the seal 108″, opening the slit 140″ to place the passage 106″ in fluid communication with the priming cap 200.

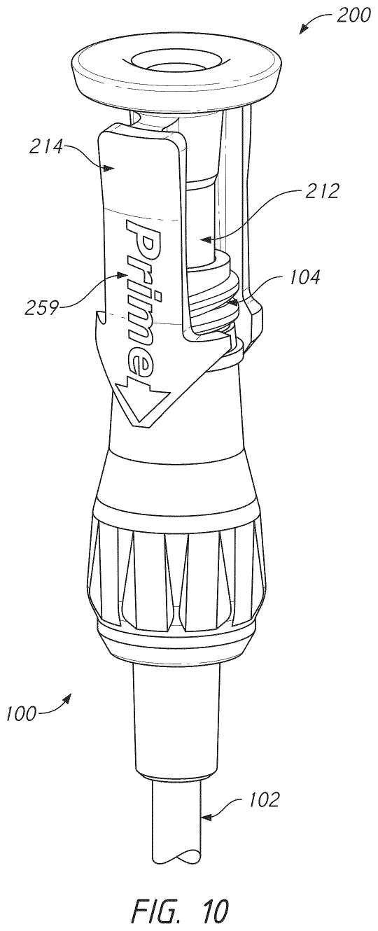

FIG. 10 is a view of the priming cap 200, according to some embodiments. Unless otherwise noted, it will be understood that any of the embodiments described and/or contemplated herein can be modified to be used with the priming cap 200 as shown in FIG. 10. As with all embodiments in this specification, any feature, structure, material, method, or step that is described and/or illustrated in the embodiment of FIG. 10 can be used with or instead of any feature, structure, material, method, or step that is described and/or illustrated in any other embodiment of this specification. In some embodiments, the resilient arms 214 can include an indicator 259 to indicate to the health care provider the proper alignment of the priming cap 200 relative to the connector 100. The indicator 259 can indicate the direction the health care provider should push the priming cap 200 during activation. In some embodiments, the indicator 259 is embossed and/or printed on the outer surface 250 of the resilient arms 214. In some embodiments, the indicator 259 is embossed and/or printed on other surfaces of the priming cap 200.

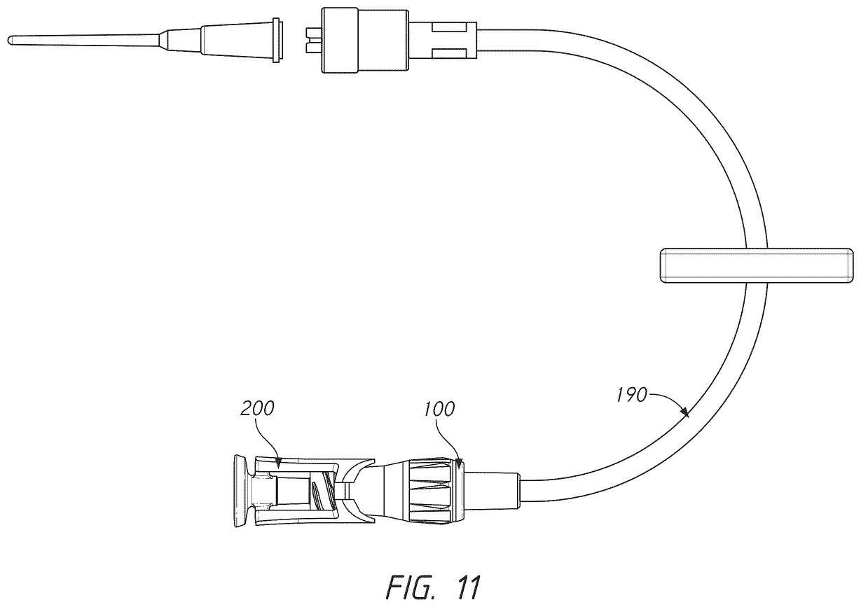

FIG. 11 illustrates an embodiment of the priming cap 200 implemented as part of an extension set 190 when in the first, inactivated position. Unless otherwise noted, it will be understood that any of the embodiments described and/or contemplated herein can perform any step and/or function discussed with reference to FIG. 11. As with all embodiments in this specification, any feature, structure, material, method, or step that is described and/or illustrated in the embodiment of FIG. 11 can be used with or instead of any feature, structure, material, method, or step that is described and/or illustrated in any other embodiment of this specification. As shown, the extension set 190 can be coupled with a catheter and/or catheter insertion device, such as a straight and/or standard safety catheter. The catheter may include a blood containment feature. In many situations, a health care provider may experience blood leakage from a rear end of the catheter device during insertion into a patient. The leakage of blood can create certain risks. For example, the leakage may be difficult to clean and/or can cause an increased risk of infection to the patient.

Implementing the priming cap 200 on the extension set 190 can provide the health care provider with the ability to quickly connect the extension set 190 to a catheter, which can help prevent or limit blood leakage. The ability to vent the extension set through the priming cap 200 can allow blood to flow into the extension set 190 more easily. This can help to limit and/or elimination trapped air from the extension set 190. Such configurations can help to limit the time to pre-prime an extension set with certain solutions or mixtures, such as saline. Thus, the priming cap 200 can enhance safety, reduce the time to establish an IV line, enhance efficiency of establishing the IV line, limit blood leakage and clean-up, reduce and/or prevent cross-contamination, reduce the risk of infection, and/or allow for easier collection of blood specimens, among other benefits and advantages.

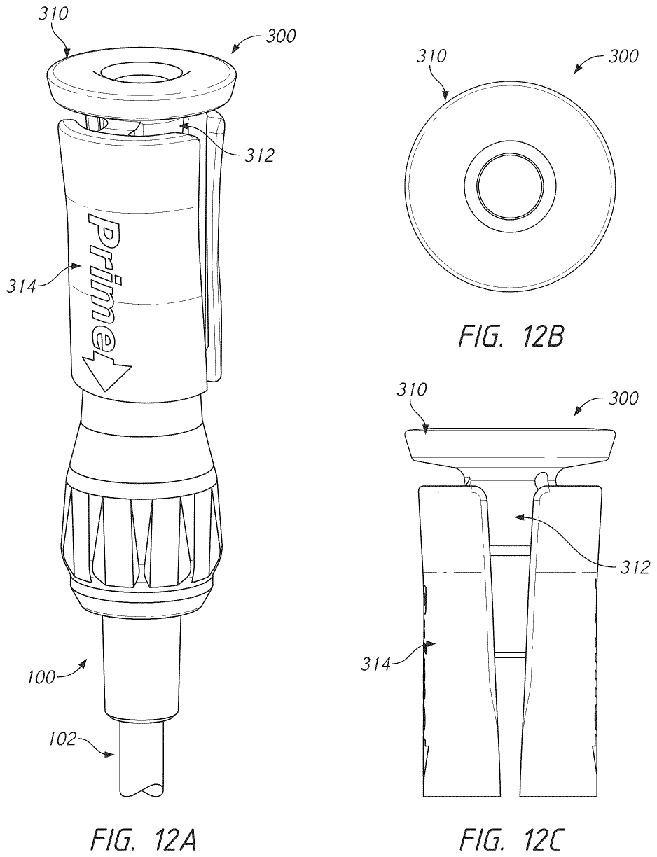

FIGS. 12A-12C are various views of a priming cap 300, according to some embodiments. In particular, FIG. 12A is a front perspective view of a priming cap 300, and FIGS. 12B and 12C are top and front views of the priming cap 300 of FIG. 12A, respectively. Unless otherwise noted, the priming cap 300 as shown in FIG. 12A may include components that are the same as or generally similar to the components in the remaining figures and/or embodiments discussed herein. It will be understood that the priming cap 300 shown in FIG. 12A can be used with any of the embodiments described and/or contemplated herein. It will also be understood that any of the embodiments described and/or contemplated herein can be modified to be used with the priming cap 300 shown in FIG. 12A. As with all embodiments in this specification, any feature, structure, material, method, or step that is described and/or illustrated in the embodiment of FIGS. 12A-12C can be used with or instead of any feature, structure, material, method, or step that is described and/or illustrated in any other embodiment of this specification.

As shown in FIGS. 12A-12C, the priming cap 300 can include resilient arms 314 that have a substantially rectangular cross-section. In some embodiments, the resilient arms 314 form a partial cylindrical shape. In some embodiments, a maximum width of an upper end of the resilient arms 314 is approximately equal to a maximum width of a lower end of the resident arms 314. For example, the maximum width of the upper and lower ends of the resilient arms 314 can be approximately uniform.

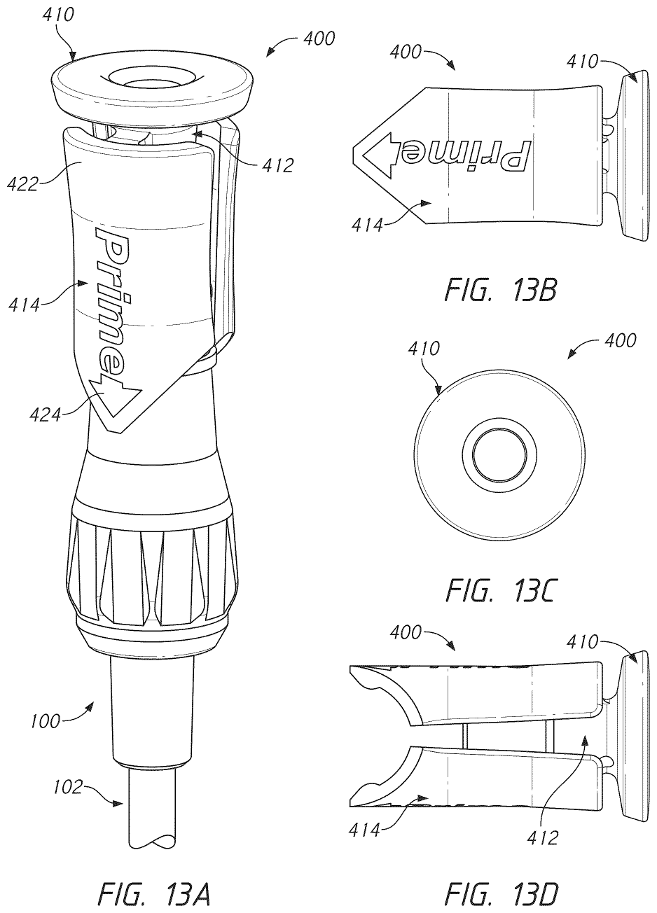

FIGS. 13A-13D are various views of a priming cap 400, according to some embodiments. In particular, FIG. 13A is a front perspective view of a priming cap 400, and FIGS. 13B-13D are side, top, and front views of the priming cap 400 of FIG. 13A, respectively. Unless otherwise noted, the priming cap 400 as shown in FIG. 13A may include components that are the same as or generally similar to the components in the remaining figures and/or embodiments discussed herein. It will be understood that the priming cap 400 shown in FIG. 13A can be used with any of the embodiments described and/or contemplated herein. It will also be understood that any of the embodiments described and/or contemplated herein can be modified to be used with the priming cap 400 shown in FIG. 13A. As with all embodiments in this specification, any feature, structure, material, method, or step that is described and/or illustrated in the embodiment of FIGS. 13A-13D can be used with or instead of any feature, structure, material, method, or step that is described and/or illustrated in any other embodiment of this specification.

As shown in FIGS. 13A-13D, the priming cap 400 can include resilient arms 414 that have a base 422 and a head 424. The base 422 can have a substantially rectangular cross-section. In some embodiments, the base 422 of the resilient arms 414 forms a partial cylindrical shape. As shown, the head 424 can transition smoothly from a side edge of the base 422. For example, a maximum lateral width of the head 424 is approximately equal to the lateral width of the base 422 at a region near the head 424. In some embodiments, a lateral width of the lower end of the head 424 is less than the maximum lateral width of the head 424 and/or the lateral width of the base 422.

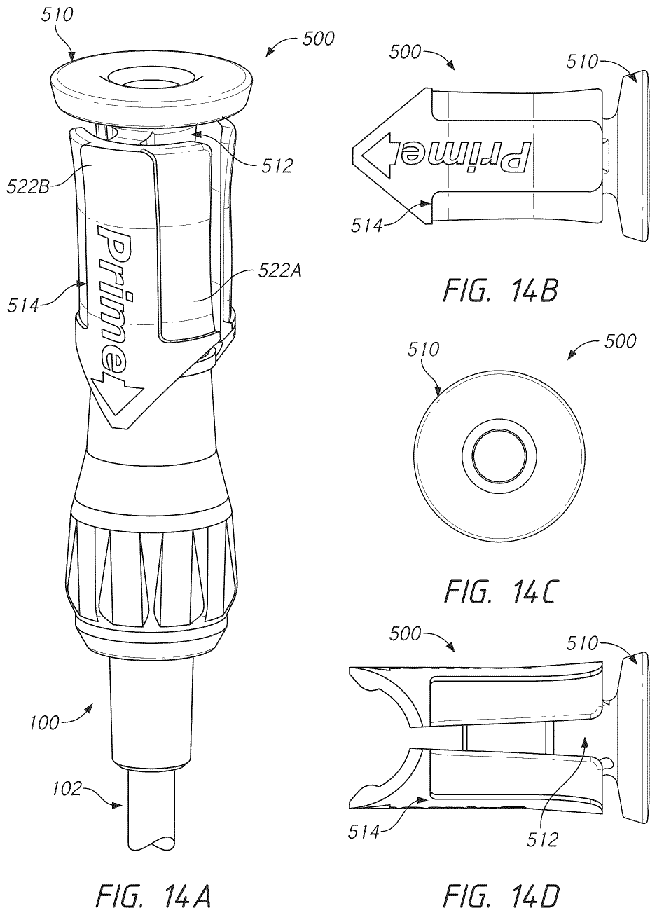

FIGS. 14-14D are various views of a priming cap 500, according to some embodiments. In particular, FIG. 14A is a front perspective view of a priming cap 500, and FIGS. 14B-14D are side, top, and front views of the priming cap 500 of FIG. 14A, respectively. Unless otherwise noted, the priming cap 500 as shown in FIG. 14A may include components that are the same as or generally similar to the components in the remaining figures and/or embodiments discussed herein. It will be understood that the priming cap 500 shown in FIG. 14A can be used with any of the embodiments described and/or contemplated herein. It will also be understood that any of the embodiments described and/or contemplated herein can be modified to be used with the priming cap 500 shown in FIG. 14A. As with all embodiments in this specification, any feature, structure, material, method, or step that is described and/or illustrated in the embodiment of FIGS. 14A-14D can be used with or instead of any feature, structure, material, method, or step that is described and/or illustrated in any other embodiment of this specification.

As shown in FIGS. 14A-14D, the priming cap 500 can include resilient arms 514 that have an inner base 522A and an outer base 522B. The inner base 522A forms a partial cylindrical shape. In some embodiments, the inner base 522A can have an approximately uniform width. In some embodiments, the inner base 522A has can have a width that is contoured. As shown, the outer base 522B can be integrally formed with the inner base 522A and/or extend outwardly from at least a portion of the inner base 522A. The outer base 522B can extend along at least a portion of an outer surface of the inner base 522A. In some embodiments, the outer base 522B is positioned near a lateral center of the inner base 522A. The outer base 522B can have a lateral width that is less than the width of the inner base 522A. In some embodiments, the outer base 522B can be integrally formed with an outer head 424. In some embodiments, at least a portion of the outer head 424 is integrally formed with and/or extends outwardly from at least a portion of the inner base 522A. In some embodiments, a maximum lateral width of the outer head 424 is larger than the width of the inner base 522A and/or the width of the outer base 522B. The outer head 424 can be respectively similar to other embodiments of the head of the resilient arms described herein.

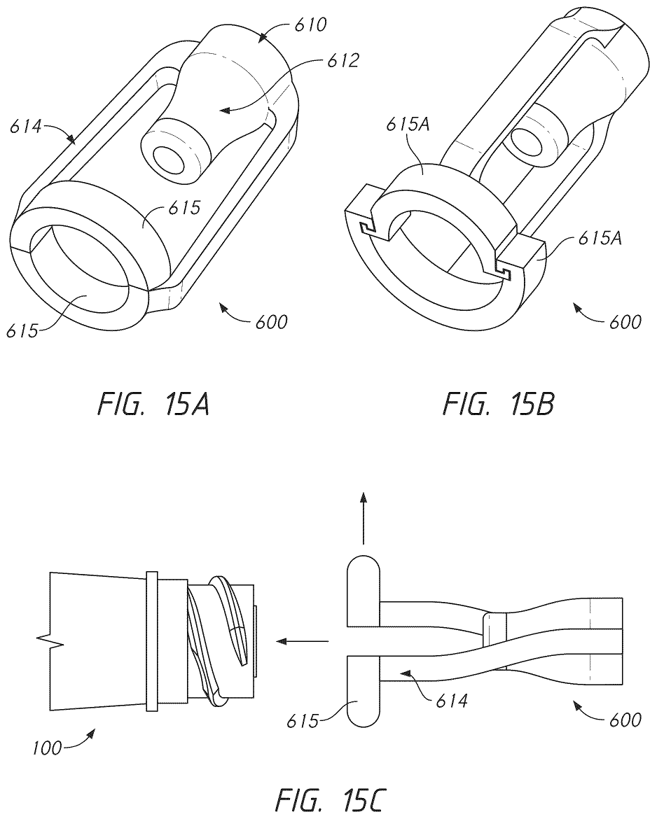

FIGS. 15A-15C are various views of a priming cap 600, according to some embodiments. In particular, FIG. 15A is a front perspective view of a priming cap 600, and FIGS. 15B and 15C are side perspective and side views of the priming cap 600 of FIG. 15A, respectively. Unless otherwise noted, the priming cap 600 as shown in FIG. 15A may include components that are the same as or generally similar to the components in the remaining figures and/or embodiments discussed herein. It will be understood that the priming cap 600 shown in FIG. 15A can be used with any of the embodiments described and/or contemplated herein. It will also be understood that any of the embodiments described and/or contemplated herein can be modified to be used with the priming cap 600 shown in FIG. 15A. As with all embodiments in this specification, any feature, structure, material, method, or step that is described and/or illustrated in the embodiment of FIGS. 15A-15C can be used with or instead of any feature, structure, material, method, or step that is described and/or illustrated in any other embodiment of this specification.

As shown in FIGS. 15A-15D, the priming cap 600 can include resilient arms 614 that extend outwardly from a portion of the conduit housing 612. The resilient arms 614 can include an engagement feature 615 configured to engage with the connector 100. For example, the engagement feature 615 can be formed on a lower end of the resilient arms 614. The engagement feature 615 can be configured to wrap around at least a portion of a circumference of the connector 100. The engagement feature 615 can form at least a portion of a cylinder. The engagement feature 615 can include a locking mechanism 615A such as a latch, a hook, and/or a hook receiver, among others. The locking mechanism 615A can engage with a corresponding locking mechanism 615A on a corresponding engagement feature 615 of another resilient arm 614. For example, the engagement feature 615 of opposite resilient arms 614 can wrap around at least a portion of the connector 100. The engagement features 615 can engage one another to lock the priming cap 600 such that the priming cap 600 is retained about at least a portion of the connector 100.

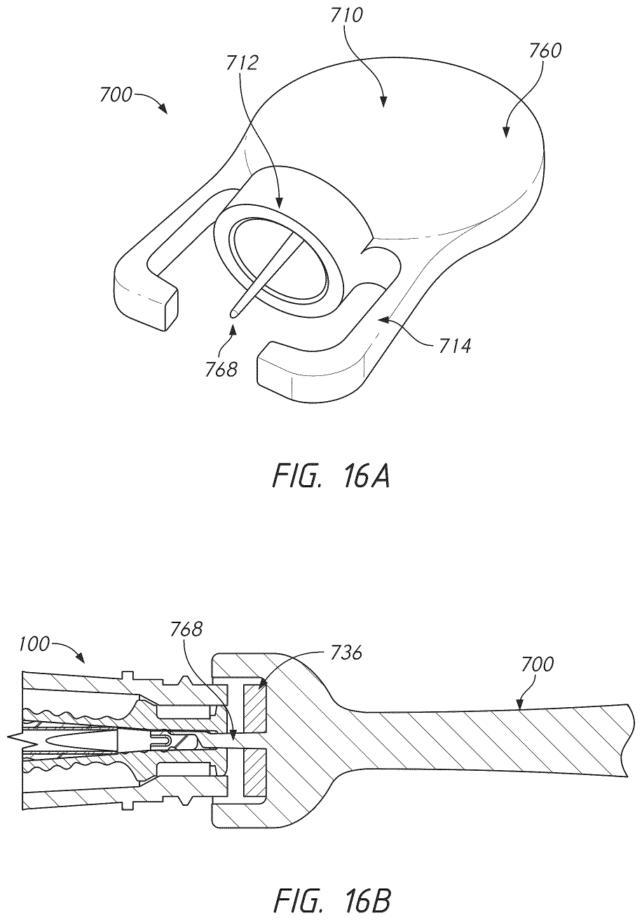

FIGS. 16A and 16B are various views of a priming cap 700, according to some embodiments. In particular, FIG. 16A is a front perspective view of a priming cap 700, and FIG. 16B is a partial side cross-sectional view of the priming cap 700 of FIG. 16A engaged to a connector 100. Unless otherwise noted, the priming cap 700 as shown in FIG. 16A may include components that are the same as or generally similar to the components in the remaining figures and/or embodiments discussed herein. It will be understood that the priming cap 700 shown in FIG. 16A can be used with any of the embodiments described and/or contemplated herein. It will also be understood that any of the embodiments described and/or contemplated herein can be modified to be used with the priming cap 700 shown in FIG. 16A. As with all embodiments in this specification, any feature, structure, material, method, or step that is described and/or illustrated in the embodiment of FIGS. 16A and 16B can be used with or instead of any feature, structure, material, method, or step that is described and/or illustrated in any other embodiment of this specification.

As shown in FIGS. 16A and 16B, the priming cap 700 can include a gripping surface 760. The gripping surface 760 can be integrally formed with at least a portion of the priming cap 700, such as the conduit housing 712 and/or the upper portion 710. The gripping surface 760 can provide an enhanced gripping area to allow the user to more easily grip the priming cap 700. As shown in the illustrated embodiment, the resilient arms 714 can be integrally formed with and/or extend outwardly from the gripping surface 760. The resilient arms 714 can define one or more tabs that can engage with a portion of the connector 100. For example, at least an end of the resilient arms 714 can contact the connector 100 with sufficient force to latch onto the connector 100 without activating the connector 100. As shown, the resilient arms 700 can spread away from the connector as the priming cap 700 is pushed into engagement with the connector 100. In some embodiments, as the resilient arms 714 spread away from the connector 100, the resilient arms 714 can be permanently and/or temporarily deformed to allow the priming cap 700 to be easily removed from the connector 100.

In some embodiments, the conduit housing 712 includes a piercing mechanism 768, such as a spike, to pierce and/or allow fluid communication between at least a portion of the connector 100 (e.g., seal 108, passage 106, and/or exit 110) and the priming cap 700. In some embodiments, as piercing mechanism 768 establishes the fluid communication, a plug 736 of the priming cap 700 may vent the connector 100 via any suitable method discussed herein.

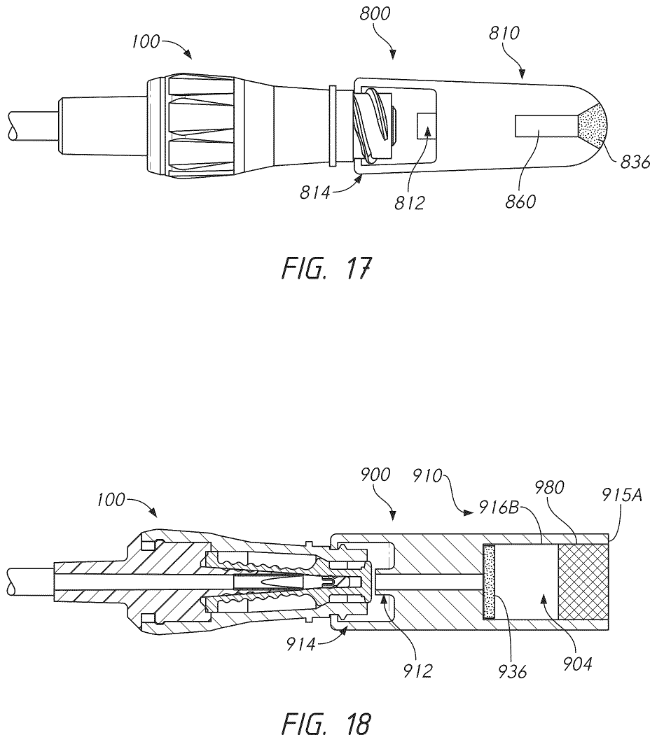

FIG. 17 is a view of a priming cap 800, according to some embodiments. In particular, FIG. 17 is a front perspective view of a priming cap 800 engaged to a connector 100. Unless otherwise noted, the priming cap 800 as shown in FIG. 17 may include components that are the same as or generally similar to the components in the remaining figures and/or embodiments discussed herein. It will be understood that the priming cap 800 shown in FIG. 17 can be used with any of the embodiments described and/or contemplated herein. It will also be understood that any of the embodiments described and/or contemplated herein can be modified to be used with the priming cap 800 shown in FIG. 17. As with all embodiments in this specification, any feature, structure, material, method, or step that is described and/or illustrated in the embodiment of FIG. 17 can be used with or instead of any feature, structure, material, method, or step that is described and/or illustrated in any other embodiment of this specification.

As shown in FIGS. 17, the priming cap 800 can include a window 860 to allow a healthcare provider to visualize a fluid flow path within the priming cap 200. The window 860 can be positioned on any portion of the priming cap 800 along the fluid passageway, such as the upper portion 810 and/or the conduit housing 812. In some embodiments, the priming cap 800 can include a plug 836 (e.g., a filter) and/or an air vent, similar to the plugs and/or air vents described herein.

FIG. 18 is a view of a priming cap 900, according to some embodiments. In particular, FIG. 18 is a front cross-sectional view of a priming cap 900 engaged to a connector 100. Unless otherwise noted, the priming cap 900 as shown in FIG. 18 may include components that are the same as or generally similar to the components in the remaining figures and/or embodiments discussed herein. It will be understood that the priming cap 900 shown in FIG. 18 can be used with any of the embodiments described and/or contemplated herein. It will also be understood that any of the embodiments described and/or contemplated herein can be modified to be used with the priming cap 900 shown in FIG. 18. As with all embodiments in this specification, any feature, structure, material, method, or step that is described and/or illustrated in the embodiment of FIG. 18 can be used with or instead of any feature, structure, material, method, or step that is described and/or illustrated in any other embodiment of this specification.

As shown in FIG. 18, in some embodiments, the priming cap 900 can include an antiseptic material 980, such as a swab and/or sponge-like material to clean at least a portion of the connector 100. In some embodiments, the antiseptic material 980 can be made of a deformable material such as foam, sponge, cross-linked matrix, gauze, cloth, woven or non-woven textile, etc. Unless otherwise noted, the antiseptic material 980 may refer to components that are the same as or generally similar to the components discussed within International Patent Publication No. WO 2018/071717, which is expressly incorporated by reference herein and made a part of this disclosure. It will be understood that the features described within the international publication can be used with any of the embodiments described and/or contemplated herein. For example, any one of the priming caps disclosed herein can be modified to include an absorbent material, as described herein and/or within the international publication.

The antiseptic material 980 can be configured to be attached to a portion of the priming cap 900. In some embodiments, the antiseptic material 980 is attached partially or entirely within the upper portion 910, such as to an inner sidewall 916B of the internal conduit 904, and/or does not attached to an inner sidewall 916B of the internal conduit 904, and/or is connected to the upper portion 910 such that at least a portion of the antiseptic material 980 overhangs or extends distally beyond an upper wall 915A of the upper portion 910. For example, as shown in FIG. 18, at least a proximal face of the antiseptic material 980 may be exposed to permit the antiseptic material 980 to interact with a portion of the connector 100, such as, for example, an end face of the connector 100. In some embodiments, the antiseptic material 980 and the upper portion 910 can be positioned and/or oriented such that their respective central longitudinal axes are generally collinear when the antiseptic material 980 is positioned on the upper portion 910.

As illustrated, a distal end face of the antiseptic material 980 can be located within the internal conduit 904 at a position spaced proximally from the plug 936, such as to form a void and/or gap between the proximal end face of the antiseptic material 980 and the plug 936. The void and/or gap may be defined so as to permit the plug 936 to properly vent the connector 100 when engaged with the connector in the activated position, as discussed herein. For example, the void and/or gap may provide sufficient volume to permit gas to pass through the plug 936 and enter the void and/or gap. In some embodiments, the antiseptic material 980 may be configured to permit the passage of gas through the antiseptic material 980.

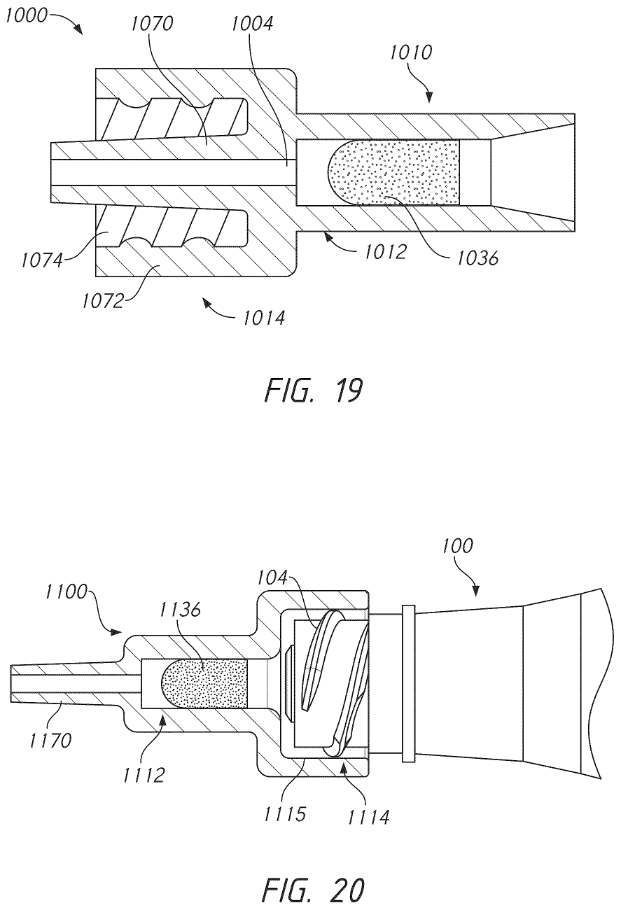

FIG. 19 is a view of a priming cap 1000, according to some embodiments. In particular, FIG. 19 is a front cross-sectional view of a priming cap 1000. Unless otherwise noted, the priming cap 1000 as shown in FIG. 19 may include components that are the same as or generally similar to the components in the remaining figures and/or embodiments discussed herein. It will be understood that the priming cap 1000 shown in FIG. 19 can be used with any of the embodiments described and/or contemplated herein. It will also be understood that any of the embodiments described and/or contemplated herein can be modified to be used with the priming cap 1000 shown in FIG. 19. As with all embodiments in this specification, any feature, structure, material, method, or step that is described and/or illustrated in the embodiment of FIG. 19 can be used with or instead of any feature, structure, material, method, or step that is described and/or illustrated in any other embodiment of this specification.

As shown, priming cap 1000 may comprise a male luer portion 1070 and a connector housing. As shown, and in some embodiments, the male luer portion 1070 may include a fluid passageway at least partially defining the internal conduit 1004. For example, at least a portion of the male luer 1070 can be inserted into at least a portion of the connector 100 to engage at least a portion of the connector 100 (e.g., seal 108) and to prime the connector 100 via any method described herein. As shown, the priming cap 1100 can include a plug 1136 (e.g., a filter) and/or an air vent, similar to the plugs and/or air vents described herein.

The connector housing illustrated in FIG. 19 may be configured to engage a needleless connector female end. In some embodiments, the connector housing includes a shroud or collar 1072 having an exterior surface (not shown) and an interior surface 1074. The interior surface 1074 can comprise a connection interface that, in some instances, can include threading. The threading of the collar 1072 may correspond to and/or be configured to engage with a corresponding feature (e.g., threading 104) on a separate connector 100. In some embodiments, the shroud or collar 1072 can be integrally formed with a male luer portion 1070.

In some embodiments, the male luer portion 1070 may be sized and configured such that the priming cap 1000 does not include a separate first, inactivated position and a second, activated position as the priming cap 1000 engages the connector 100. In such instances, the priming cap 1000 and the connector 100 may only include one activated position. For example, as priming cap 1000 is threaded onto connector 100, the male luer portion 1070 may automatically engage at least a portion of the connector 100 (e.g., the seal 108) such that the connector 100 begins transitioning from the opened position to the closed position. In this manner, the priming cap 1000 may begin venting the connector 100 once the priming cap 1000 engages the connector 100.

FIG. 20 is a view of a priming cap 1100, according to some embodiments. In particular, FIG. 20 is a front cross-sectional view of a priming cap 1100 engaged to a connector 100. Unless otherwise noted, the priming cap 1100 as shown in FIG. 20 may include components that are the same as or generally similar to the components in the remaining figures and/or embodiments discussed herein. It will be understood that the priming cap 1100 shown in FIG. 20 can be used with any of the embodiments described and/or contemplated herein. It will also be understood that any of the embodiments described and/or contemplated herein can be modified to be used with the priming cap 1100 shown in FIG. 20. As with all embodiments in this specification, any feature, structure, material, method, or step that is described and/or illustrated in the embodiment of FIG. 20 can be used with or instead of any feature, structure, material, method, or step that is described and/or illustrated in any other embodiment of this specification.

As shown in FIG. 20, the priming cap 1100 can include a luer slip lock configuration. For example, the resilient arms 1114 can include engagement features to conform to corresponding features, such as threads 104, of the connector 100. In some embodiments, the resilient arms 1114 can be integrally formed with a male luer portion 1170. For example, the resilient arms 1114 can be disengaged from the connector 100. Once the resilient arms 1114 are disengaged, the orientations of the priming cap 1100 can be reversed and at least a portion of the male luer 1170 can be inserted into at least a portion of the connector 100 to engage at least a portion of the connector 100 (e.g., seal 108) and to prime the connector 100 via any method described herein. As shown, the priming cap 1100 can include a plug 1136 (e.g., a filter) and/or an air vent, similar to the plugs and/or air vents described herein.

As illustrated in FIG. 20, the resilient arms 1114 may comprise a resilient shroud or collar configured to engage a needleless connector female end. For example, in some embodiments, the resilient shroud 1114 can comprise an interior surface 1115 configured to interact with a portion of the connector 100, such as, for example, an end portion of the connector 100, the threads 104 of the connector 100, and/or one or more features of the connector 100, among others. In some embodiments, as illustrated, the interior surface 1115 can be threadless and have any suitable surface texture, such as, for example, smooth and/or rough. In some embodiments, the resilient shroud 1114 can be pushed and/or twisted onto and/or off a portion of the connector 100. In some embodiments, a diameter of the interior surface 1115, as shown in FIG. 20, may be essentially the same size as a diameter of an outer surface (e.g., threads 104) of the connector 100 to facilitate interaction between the priming cap 1100 and the connector 100. For example, at least a portion of the interior surface 1115 can be configured to slidably contact a portion of the connector 100, thereby urging the interior surface 1115 to resiliently or elastomerically expand or stretch or otherwise move to receive the portion of the connector 100 for attachment.

To facilitate attaching the resilient shroud 1114 to the connector 100, in some embodiments, the priming cap 1100 can comprise a semi-rigid material capable of deformation when a load is applied. This can advantageously allow the interior surface 1115 of the resilient shroud 1114 to temporarily and/or permanently deform when the interior surface 1115 interacts with one or more features of the connector 100 (e.g., thread 104). In some embodiments, the priming cap 1100 can comprise a rigid material that is sufficiently pliable to permit the interior surface 1115 to engage with a portion of the connector 100. In some embodiments, the interface between the connector 100 and the priming cap 1100 can form a fluid tight seal. It will be appreciated that the ability of the interior surface 1115 to deform can advantageously allow the priming cap 1100 to be removably attached to the connector 100 without the use of threads. For example, in some embodiments, the semi-rigid material can be configured to allow threads 104 of the connector 100 to slide into the resilient shroud 1114 (as shown in FIG. 20) such that the interior surface 1115 deforms radially outward as the threads 104 interact with the interior surface 1115 when they are sliding in. In some embodiments, the interior surface 1115 can be configured to rebound radially inward after the threads 104 interact with the interior surface 1115 and slide further into the resilient shroud 1114. When the connector 100 is fully inserted into the priming cap 1100, the interior surface 1115 can be configured to deform radially outward wherever the connector 100 interacts with the interior surface 1115. Advantageously, a threadless resilient shroud 1114 can be configured to receive one or more connectors having one or more different features (e.g., various thread characteristics, differently sized connectors, among others).

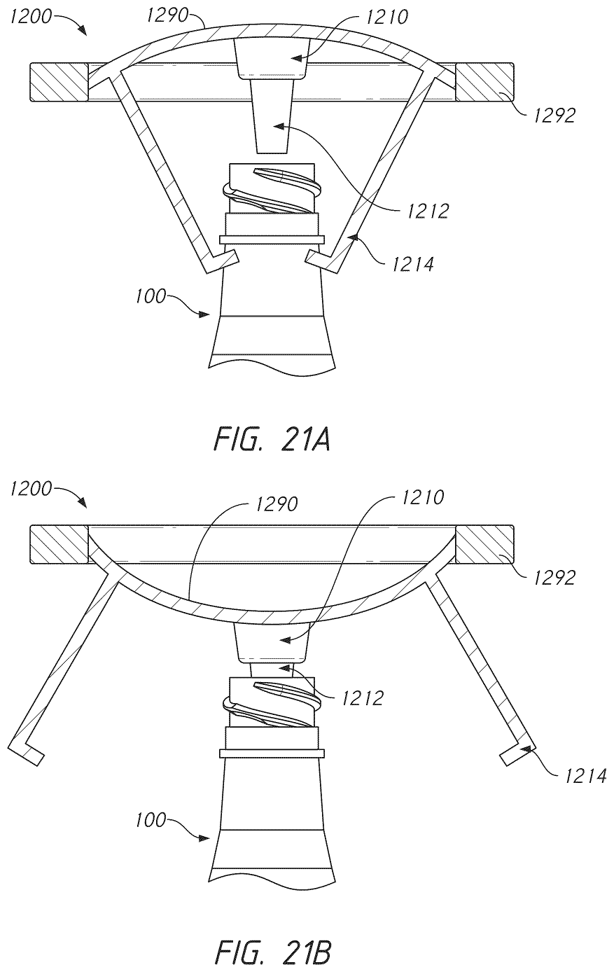

FIGS. 21A and 21B are various views of a priming cap 1200, according to some embodiments. In particular, FIG. 21A is a front view of a priming cap 1200 engaged to a connector 100 in a first, inactivated position, and FIG. 21B is a front view of the priming cap 1200 and the connector 100 of FIG. 21A in a second, activated position. Unless otherwise noted, the priming cap 1200 as shown in FIGS. 21A and 21B may include components that are the same as or generally similar to the components in the remaining figures and/or embodiments discussed herein. It will be understood that the priming cap 1200 shown in FIG. 21A can be used with any of the embodiments described and/or contemplated herein. It will also be understood that any of the embodiments described and/or contemplated herein can be modified to be used with the priming cap 1220 shown in FIG. 21A. As with all embodiments in this specification, any feature, structure, material, method, or step that is described and/or illustrated in the embodiment of FIG. 21A can be used with or instead of any feature, structure, material, method, or step that is described and/or illustrated in any other embodiment of this specification.

As shown in FIGS. 21A and 21B, the priming cap 1200 can include an upper portion 1210, a conduit housing 1212, and resilient arms 1214 that are integrally formed with a deformable portion 1290. The deformable portion 1290 can be moveable secured to a rigid ring 1292. The resilient arms 1214 can be substantially similar to any resilient arms described herein. For example, at least an end of the resilient arms 1214 can contact the connector 100 with sufficient force to latch onto the connector 100 without activating the connector 100.

In some embodiments, the resilient arms 1214 can spread away from the connector 100 as the conduit housing 1212 of the priming cap 1200 is pushed into engagement with the connector 100. In some embodiments, as the conduit housing 1212 is pushed towards the connector 100 from the first, inactivated positions to the second, activated position, the deformable portion 1290 can pivot about the rigid ring 1292. For example the deformable portion 1290 pivots and/or deforms from a first position in which the deformable portion 1290 is concave (see FIG. 21A) to a second position in which the deformable portion 1290 is convex (see FIG. 21B). As the deformable portion 1290 moves from the first position to the second position, the conduit housing 1212 is pushed into engagement with the connector 100 to activate and/or vent the connector 100 (as discussed herein). This can cause the resilient arms 1214 to spread away from the connector 100. In some embodiments, as the resilient arms 1214 spread away from the connector 100, the resilient arms 1214 can be permanently and/or temporarily deformed to allow the priming cap 1200 be easily removed from the connector 100. In some embodiments, as the resilient arms 1214 spread away from the connector 100, the resilient arms 1214 may break away from the priming cap 1200, allowing the priming cap 1200 to be easily removed from the connector 100.

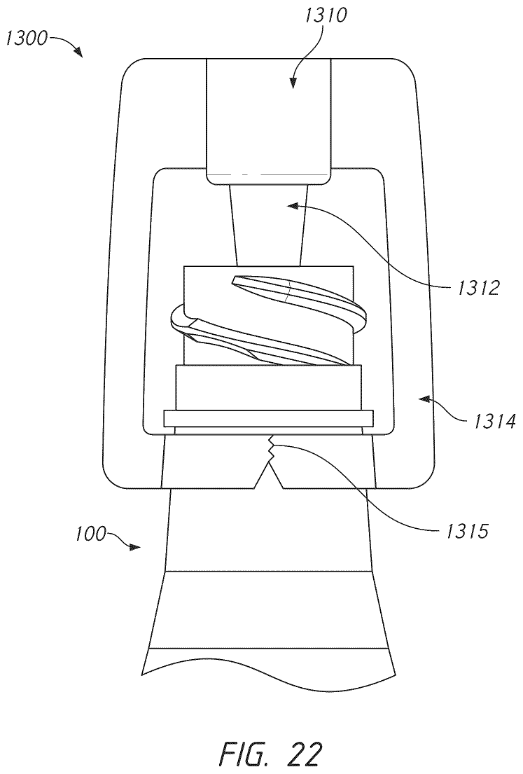

FIG. 22 is a view of a priming cap 1300, according to some embodiments. In particular, FIG. 22 is a front cross-sectional view of a priming cap 1300 engaged to a connector 100. Unless otherwise noted, the priming cap 1300 as shown in FIG. 22 may include components that are the same as or generally similar to the components in the remaining figures and/or embodiments discussed herein. It will be understood that the priming cap 1300 shown in FIG. 22 can be used with any of the embodiments described and/or contemplated herein. It will also be understood that any of the embodiments described and/or contemplated herein can be modified to be used with the priming cap 1300 shown in FIG. 22. As with all embodiments in this specification, any feature, structure, material, method, or step that is described and/or illustrated in the embodiment of FIG. 22 can be used with or instead of any feature, structure, material, method, or step that is described and/or illustrated in any other embodiment of this specification.

As shown in FIG. 22, the priming cap 1300 can comprise a structure for preventing the priming cap 1300 from easily disengaging from the connector 100 when in the first, inactivated position. The resilient arms 1314 can include an engagement feature 1315 configured to, at least initially, prevent or impede the disengagement of the priming cap 1300 from the connector 100. In some embodiments, the priming cap 1300 may be manufactured on the connector 100 with the priming cap 1300 and the connector 100 in the first, inactivated position and the engagement feature 1315 intact. Thus, the priming cap 1300 and/or the connector 100 can be assembled in the first, inactivated position before use (e.g., during shipment and/or packaging, or before priming).

The engagement feature 1315 may be configured to engage at least one of the resilient arms 1314 with another of the resilient arms 1314, as illustrated in FIG. 22, when the priming cap 1300 is in the first, inactivated position with the connector 100. For example, the engagement feature 1315 can be formed on a lower end of the resilient arms 1314. The engagement feature 1315 can be configured to wrap around at least a portion of a circumference of the connector 100. For example, at least a portion of the engagement feature 1315 can wrap around at least a portion of the connector 100. The engagement feature 1315 can engage and/or lock the priming cap 1300 such that the priming cap 1300 is retained about at least a portion of the connector 100 in the first, inactivated position, as described herein.

In some embodiments, as the priming cap 1300 and the connector 100 transition from the first, inactivated position towards the second, activated positions (as described herein), the resilient arms 1314 may move distally along the connector 100 causing the resilient arms 1314 to spread away from the connector 100 and each other. In some instances, as the resilient arms 1314 spread away from each other, the engagement feature 1315 can be configured to shear or break before as the priming cap 1300 and the connector 100 transition to the second, activated position. The engagement feature 1315 may advantageously be configured to inhibit removal of the priming cap 200 from the connector 100 until the priming cap 200 and the connector 100 transition to the second, activated position. Once the engagement feature 1315 shears or breaks, the priming cap 1300 may be easily removed from the connector 100.

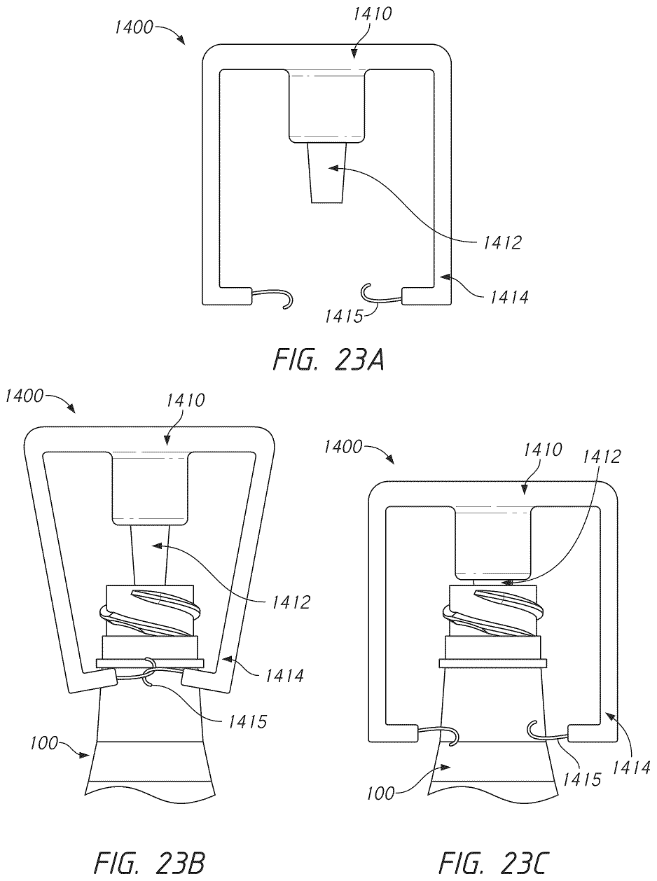

FIGS. 23A-23C are various views of a priming cap 1400, according to some embodiments. In particular, FIG. 23A is a front view of a priming cap 1200, and FIGS. 23B and 23C are a front views of the priming cap 1400 of FIG. 23A engaged to a connector 100 in a first, inactivated position and in a second, activated position, respectively. Unless otherwise noted, the priming cap 1400 as shown in FIGS. 23A-23C may include components that are the same as or generally similar to the components in the remaining figures and/or embodiments discussed herein. It will be understood that the priming cap 1400 shown in FIG. 23A can be used with any of the embodiments described and/or contemplated herein. It will also be understood that any of the embodiments described and/or contemplated herein can be modified to be used with the priming cap 1400 shown in FIG. 23A. As with all embodiments in this specification, any feature, structure, material, method, or step that is described and/or illustrated in the embodiment of FIG. 23A can be used with or instead of any feature, structure, material, method, or step that is described and/or illustrated in any other embodiment of this specification.

As shown in FIGS. 23A-23C, the priming cap 1400 can include resilient arms 1414 that are substantially similar to the resilient arms described above with respect to the priming cap 600. For example, at least an end of the resilient arms 1414 can include an engagement feature that contacts the connector 100 with sufficient force to latch onto the connector 100 in a first position without activating the connector 100. In some embodiments, the resilient arms 1414 include a locking mechanism 1415 such as a latch, notch, clip, hook, and/or a snapping feature, among others. The locking mechanism 1415 of one resilient arm 1414 can engage the locking mechanism of another resilient arm 1414 to surround and/or engage a portion of the connector 100 and/or to secure the priming cap 1400 to the connector 100. As the conduit housing 1412 is pushed into engagement with the connector 100, the resilient arms 1414 spread and/or spring away from one another, causing the locking mechanism 1415 to disengage or break, and allowing the priming cap 1400 to be easily removed.

Although this invention has been disclosed in the context of certain embodiments and examples, it will be understood by those skilled in the art that the present invention extends beyond the specifically disclosed embodiments to other alternative embodiments and/or uses of the invention and obvious modifications and equivalents thereof. In addition, while several variations of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. It should be understood that various features and aspects of the disclosed embodiments can be combined with, or substituted for, one another in order to form varying modes of the disclosed invention. Thus, it is intended that the scope of the present invention herein disclosed should not be limited by the particular disclosed embodiments described above, but should be determined only by a fair reading of the claims that follow.

The following is claimed:

1. A priming cap for use with a medical connector including a valve at a first end of the medical connector configured to selectively close a fluid path in the medical connector, the priming cap comprising:
a valve activation member configured to selectively engage the valve of the medical connector to open the fluid path in the medical connector,
a permeable portion configured to permit the passage of air through the priming cap and configured to inhibit the passage of liquid through the priming cap, and
an engagement member configured to selectively secure the priming cap to the medical connector, wherein the engagement member is configured to secure the priming cap in a first position in which the fluid path in the medical connector is closed and a second position in which the fluid passage in the medical connector is open.

2. The priming cap of claim, wherein the engagement member includes a bumper that extends radially inwardly from an interior surface of the engagement member, and wherein the bumper is configured to secure the priming cap to a securement feature of the medical connector.

3. The priming cap of claim 1, wherein the valve activation member is positioned adjacent to the medical connector when the priming cap is in the first position.

4. The priming cap of claim 3, wherein the valve activation member is at least partially positioned within the medical connector when the priming cap is in the first position.

5. The priming cap of claim, wherein the permeable member is a hydrophobic plug positioned in a fluid flow path extending through the valve activation member.

6. The priming cap of claim 5, wherein the valve activation member comprises a lower region and an upper region, wherein a transition region between the upper and the lower regions defines a platform configured to support the plug within the valve activation member fluid flow path.

7. The priming cap of claim, wherein the engagement member comprises at least two resilient arms.

8. The priming cap of claim 7, wherein each of the at least two resilient arms comprises a base portion and a head portion, wherein the base portion and the head portion are integrally formed and shaped in an arrow configuration, wherein the arrow configuration is configured to indicate to a user a proper orientation of the priming cap relative to the connector.

9. The priming cap of claim 8, wherein the head portion is configured to be closer to the medical connector when the priming cap is secured to the medical connector than the base portion.

10. The priming cap of claim 7, wherein the at least two resilient arms extend from the valve activation member and are radially spaced away from a lower region of the valve activation member.

11. The priming cap of claim 7, wherein each resilient arm further comprises an indicator.

12. The priming cap of claim, wherein the priming cap further comprises a window configured to allow visualization of at least a portion of the fluid passageway of the valve activation member.

13. The priming cap of claim further comprising an antiseptic material.

14. The priming cap of claim 13, wherein the antiseptic material is at least partially located within the priming cap and is configured to interact with the medical connector.

15. The priming cap of claim 14, wherein the antiseptic material is located proximal to the plug.

16. An automatic closed priming system comprising:
the priming cap of claim and
the medical connector.

17. The automatic closed priming system of claim 16, wherein the medical connector includes tubing extending from a second end of the medical connector.

18. A method of priming a medical connector including a valve at a first end of the medical connector configured to selectively close a fluid path in the medical connector, the method comprising:
providing a pre-assembled priming cap and medical connector assembly, the priming cap including:
a valve activation member configured to selectively engage the valve of the medical connector to open the fluid path in the medical connector,
a permeable portion configured to permit the passage of air through the priming cap and configured to inhibit the passage of liquid through the priming cap, and
an engagement member configured to selectively secure the priming cap to the medical connector;
wherein the pre-assembled the priming cap and medical connector assembly includes the priming cap attached to the medical connector in a first position in which the valve activation member of the priming cap does not activate the valve of the needleless medical fluid connector female end to open the connector; and
transitioning the priming cap from the first position to a second position in which the valve activation member of the priming cap activates the valve of the medical connector to establish fluid communication between the medical connector and the priming cap, and
permitting fluid to flow through the medical connector and into the priming cap.

19. The method of priming a medical connector of claim 18, wherein the valve activation member of the priming cap further includes a fluid flow path and the step of transitioning the priming cap from the first position to the second position includes establishing fluid communication between the fluid path of the medical connector and the fluid path of the priming cap.
